(19) United States
(12) Patent Application Publication
(10) Pub. No.: US 2019/0284621 A1
(43) Pub. Date: Sep. 19, 2019

(54) THERAPEUTIC OLIGONUCLEOTIDES CAPTURE AND DETECTION

(71) Applicant: Roche Innovation Center Copenhagen A/S, Hørsholm (DK)

(72) Inventors: Mads Aaboe JENSEN, Farum (DK); Lars JOENSON, Viby Sjaelland (DK); Lukasz KIELPINSKI, Frederiksberg (DK); Morten LINDOW, Copenhagen SV (DK); Jonas VIKESAA, Kirke Hyllinge (DK)

(21) Appl. No.: 16/349,250

(22) PCT Filed: Nov. 9, 2017
(86) PCT No.: PCT/EP2017/078695

§ 371 Date: May 10, 2019

(30) Foreign Application Priority Data
Nov. 11, 2016 (EP) 16198340.8

(51) Int. Cl. C12Q 1/6862; C12Q 1/6855; C12Q 1/6869

(52) U.S. Cl. CPC C12Q 1/6862; C12Q 1/6855; C12Q 1/6869; C12Q 2521/501; C12Q 2545/114; C12Q 2525/155; C12Q 2525/186; C12Q 2525/301; C12Q 2525/117

(57) ABSTRACT

The present invention relates to the detection of therapeutic modified oligonucleotides in biological samples and an adaptor oligonucleotide (capture probe) which enables a quantitative PCR based detection method and the sequencing of modified oligonucleotides. The invention provides novel adaptor probes for use in detecting therapeutic oligonucleotides and for in vivo discovery of preferred therapeutic oligonucleotide sequences.

### FIELD OF INVENTION

The present invention relates to the detection of therapeutic modified oligonucleotides in biological samples and an adaptor oligonucleotide (capture probe) which enables a quantitative PCR based detection method and the sequencing of stereodefined or sugar-modified oligonucleotides. The invention provides novel adaptor probes for use in detecting therapeutic oligonucleotides and for in vivo discovery of preferred therapeutic oligonucleotide sequences.

### BACKGROUND

Modified oligonucleotides, such as antisense oligonucleotides, siRNAs and aptamers are being developed as therapeutic agents. The qualitative and quantitative detection of these oligonucleotides in samples like cell cultures, tissue, blood, plasma or urine is a prerequisite to assess their use and to monitor their intracellular uptake, bio-distribution, metabolism and stability in vivo and/or in vitro.

Therapeutic modified oligonucleotide assert their function when delivered to target cells and tissues. However, upon delivery of such therapeutic oligonucleotide, it is often difficult to determine the level of exposure as the extent of oligonucleotide uptake varies in cells and tissues. As a result, accurately determining an effective dose of a therapeutic oligonucleotide can be challenging. Current methods to measure oligonucleotide exposure in target cells and tissues include either MS-based or ELISA-like methods, which are routinely used in the development and clinical evaluation of therapeutic oligonucleotides. The sensitivity of those methods is in the range of nM to pM concentration for detection of oligonucleotides and the assay development time can be several months. Furthermore, MS- and ELISA-like methods will only be able to measure a single antisense oligonucleotide per sample. Here, we present a PCR-based method that is 1) orders of magnitude more sensitive compared to current methods, 2) has a short development time (weeks) and 3) will allow evaluation of multiple oligonucleotides per sample. Further advantages are disclosed herein and are illustrated in the examples.
WO99/14226 discloses the use of [alpha³²P] ddNTP's and ThermoSequenase™ DNA Polymerase to Sequence DNA Templates Containing LNA T Monomers.
Shiraki et al., (2003) PNAS Vol 100, 15776-15781 discloses double stranded capture probes used in cap analysis gene expression (CAGE).
Zhao et al., Acta Biochim Biophys Sin (2014) 46 (9): 727-737, reports on the effects of 2′-O-methyl nucleotides on ligation capability of T4 DNA ligase.
WO2014/110272 discloses compositions and methods for ligating single stranded nucleic acids with an acceptor molecule which comprises a hairpin structure and a 3′ overhang for oligonucleotide capture.
WO2008/046645 discloses a capture probe-PCR based method using LNA capture probe which was used in '645 to capture a DNA phosphorothioate oligonucleotide (G3139), followed by a quantitative PCR step. This method appears to be based upon known methods for detecting and cloning unmodified oligonucleotides such as microRNAs. However, WO'645 alleges that the method can be used for sugar modified oligonucleotides such as LNA or MOE compounds.

However, as is detailed herein, such traditional cloning methods do not function effectively for sugar modified oligonucleotides such as LNA or MOE, this is thought to be due to the lack of effective chain elongation from terminal modified nucleosides, as well as an inhibitory effect of modified nucleoside templates, particularly high affinity nucleoside templates, on DNA polymerases.

### OBJECTIVE OF THE INVENTION

The present invention provides an enhanced capture probe for use in the detection, quantification, sequencing, amplification, or cloning of a sugar modified oligonucleotides and stereodefined oligonucleotide, such as an LNA modified oligonucleotides. The method also provides a method of capturing, detecting, quantifying, amplifying, and cloning nucleoside modified oligonucleotides and stereodefined oligonucelotides.

The present invention overcomes obstacles in applying qPCR to the detection of modified oligonucleotides. This has been achieved by employing a uniquely designed oligonucleotide capture probe combined with a T4DNA ligase step prior to chain elongation.

### SUMMARY OF INVENTION

The invention provides for a capture probe oligonucleotide, comprising 5′-3′;
i) a first nucleotide segment comprising
   a. at least 3 5′ contiguous nucleotides of predetermined sequence (1A), wherein the 5′ most nucleotide is a DNA nucleotide with a terminal 5′ phosphate group;
   b. optionally a region of degenerate or predetermined nucleotides, positioned 3′ of region 1A (1B);
   c. a 3′ region which comprises a universal primer binding site [a predetermined region of nucleotides] (1C);
ii) a second nucleotide segment, comprising
   a. a contiguous sequence of nucleotides which are complementary to the predetermined sequence 1A of the first segment (2A);
   b. a region of at least 2 nucleotides, wherein the 3′ most nucleotide is a terminal nucleotide with a blocked 3′ terminal group (2B);
   wherein the first and second regions are covalently linked via a polymerase blocking linker moiety.

See FIG. 14 for a general outline of the design of the capture probe.

The capture probe may be for use in or detecting, quantifying, amplify, sequencing or cloning a nucleoside modified oligonucleotide. The capture probe may be for use in or detecting, quantifying, amplify, sequencing or cloning an oligonucleotide which comprises a Rp phosphorothioate internucleoside linkage between the two 3′ terminal nucleosides on the oligonucleotide.

The invention provides for the use of the capture probe oligonucleotide for use in detecting, quantifying, sequencing, amplifying or cloning a nucleoside modified oligonucleotide.

The invention provides for the use of the capture probe oligonucleotide for use in detecting, quantifying, sequencing, amplifying or cloning an oligonucleotide which comprises an Rp phosphorothioate internucleoside linkage between the two 3′ terminal nucleosides on the oligonucleotide.

The invention provides for the use of T4DNA ligase to ligate the 3′ terminus of a nucleoside modified oligonucleotide to the 5′ terminus of a DNA oligonucleotide, wherein the 3′ nucleoside of the nucleoside modified oligonucleotide is a LNA nucleoside.

The invention provides for the use of T4DNA ligase to ligate the 3′ terminus of a stereodefined oligonucleotide to the 5′ terminus of a DNA oligonucleotide, wherein the stereodefined oligonucleotide comprises a Rp phosphorothioate internucleoside linkage between the two 3′ terminal nucleosides on the oligonucleotide.

In some embodiments of the methods or uses of the invention, the ligation between the 3′ terminus of the modified oligonucleotide and the 5′ terminus of a DNA oligonucleotide, such as the capture probe, is performed in the presence of a polyethyleneglocol polymer, such as PEG 4000. The concentration of the PEG polymer, such as PEG 4000 is, in some embodiments between about 10% and about 30%, such as between about 12% and about 25%, such as between about 15% and about 20%, such as about 15% or about 20%.

The invention provides for a method for detecting, quantifying, amplifying, sequencing or cloning a nucleoside modified oligonucleotide in a sample, said method comprising the steps of;
a. optionally perform an RNase and/or DNAase treatment of the sample,
b. admixing a capture probe oligonucleotide and the sample under conditions which allow hybridization of the capture probe oligonucleotide to the nucleoside modified oligonucleotide,
c. performing T4 DNA ligase mediated ligation of the 5′ terminus of the capture probe oligonucleotide and the 3′ terminus of the nucleoside modified oligonucleotide,
d. add a universal primer which is complementary to a part of the capture probe oligonucleotide,
e. perform chain elongation of the universal primer in the presence of a DNA polymerase or reverse transcriptase,
f. detect, quantify, sequence or clone the chain elongation product obtained in step e).

The sample may for example be a purified nucleic acid fraction, obtained from a biological sample, such as a patient sample.

The invention provides for a method for detecting, quantifying, sequencing or cloning a nucleoside modified oligonucleotide in a sample, said method comprising the steps of;
a. optionally perform an RNase and/or DNase treatment of the sample,
b. admixing the capture probe oligonucleotide of the invention and the sample under conditions which allow hybridization of region 2B of the capture probe oligonucleotide to the nucleoside modified oligonucleotide,
c. performing T4 DNA ligase mediated ligation of the 5′ terminus of the capture probe oligonucleotide and the 3′ terminus of the nucleoside modified oligonucleotide,
d. add a universal primer which is complementary to region 1A of the capture probe oligonucleotide,
e. perform chain elongation of the universal primer in the presence of a DNA polymerase or reverse transcriptase,
f. detect, quantify, sequence or clone the chain elongation product obtained in step e).

The invention provides for a method for identifying a nucleoside modified oligonucleotide which is enriched in a target cell or tissue, e.g. in a mammal or a human, said method comprising;
a. administering a mixture of nucleoside modified oligonucleotides with different nucleobase sequences to a mammal,
b. allow for the oligonucleotides to be distributed within the mammal, for example for a period of at least 24-48 hours,
c. isolate a population of modified oligonucleotides from the target cell or tissue of the mammal,
d. perform the method according to the invention, including the step of sequencing the population of modified oligonucleotides, to
e. identify nucleoside modified oligonucleotide sequences which are enriched in the target tissue of the mammal.

### BRIEF DESCRIPTION OF FIGURES

FIG. 1: An attempt of ligating three different, labeled capture probe oligonucleotides with pool of three LNA containing oligonucleotides using two different enzymes. None of the capture probe oligonucleotides-enzyme combinations yielded detectable ligation product.

FIG. 2: (Relevant for examples 1 and 2). Design of capture probe oligonucleotides used in the study.

FIG. 3: An attempt of ligating four different, labeled capture probe oligonucleotides utilizing overhang concept with a randomized pool of LNA containing oligonucleotides using four different enzymes. Some enzyme-adapter combinations yielded detectable ligation product as indicated by the appearance of a band migrating slower than the adapter (T4 DNA Ligase+a4; T7 DNA Ligase+a4; T4 DNA Ligase+a5).

FIG. 4: Further characterization of the successful ligation reaction. Reaction 1—reduced adapter a4 concentration, reaction 2—reference reaction, reaction 3—increased LNA oligonucleotide concentration, reaction 4—reaction with heat inactivated T4 DNA Ligase, reaction 5—reaction without T4 DNA Ligase.

FIG. 5: Impact of the concentration of adapter on the yield of ligated product. Reactions labeled “H” contained 1 μM LNA oligonucleotide, reactions labeled “L” contained 0.2 μM LNA oligonucleotide. Number following the character indicates the concentration of a4 adapter in the final reaction (in μM).

FIG. 6. Impact of the time of ligation on the yield of ligated product. Reactions labeled “H” contained 1 μM LNA oligonucleotide, reactions labeled “L” contained 0.2 μM LNA oligonucleotide. Number following the character indicates number of ligation cycles underwent by the sample.

FIG. 7. Impact of the concentration of PEG 4000 on the yield of ligated product. Number indicates the concentration of PEG 4000 in the final reaction.

FIG. 8: Sybr Green qPCR reaction on LNA1 ligation product. Sybr Green qPCR reaction was performed on dilutions of the ligation between O6 and O6-CP1. Ligation was performed using an input of 100 μM O6 and 100 μM of O6-CP1 in the presence or absence of T4-DNA-Ligase. The ligation mix was diluted to 250 μM, 62.5 μM, 15.63 μM, 3.91 μM, 976 fM, 244 fM, 61 fM and 2 μl was used as input in a 10 μl PCR reaction with a technical replicate. The PCR reaction was performed on a Viia7 qPCR machine using Sybr Green qPCR chemistry with the O6-p1 and Cp1-p1 primers. 8A displays the qPCR curve of the different concentration of input material with the fluorescent intensity display at the Y-axis as a function of the PCR cycle number. 8B displays the same reactions except T4 DNA ligase wasn't present during the ligation between O6 and O6-CP1.

FIG. 9: Linearity of LNA-DNA ligation reaction. A 10× dilution curve of LNA-mix-pool1 (see MM) (1 nM, 100 pM, 10 pM, 1 pM, 100 fM, 10 fM, 1 fM) or H₂O was used as input for a LNA-DNA ligation reaction using LNA1-capture probe (10 nM) in the presence or absence of T4 DNA ligase. Panel 9A displays Sybr Green PCR curves on the ligations using O6-p2 and CP1-p1. 9B Graph depicts the measured amount of material (2^{-Ct}*10¹²) vs the actual input amount. A power regression trend line was calculated to depict the linearity and efficiency of the reaction. Panel 9C displays Sybr Green PCR curves on the ligations using the primers O6-p1 and CP1-p1 on the ligation reaction were T4 DNA ligase was not present.

FIG. 10: qLNA-PCR specificity. LNA ligation was performed using LNA specific capture probes on dilutions of LNA-mix-pool1. LNA-mix-pool1 was prepared by dilution to 1 nM 200 pM 40 pM 8 pM 1.6 pM 320 fM 64 fM of each individual oligo or pure H₂O and 2 μl of this was as input in 20 μL ligation reactions. Each ligation reaction was performed in the presence or absence of T4-DNA ligase. The ligation reaction was diluted 9× before 2 μl was used as input in a Sybr Green qPCR reaction. Panel 10A shows technical replicates of qPCR reactions from the ligation reaction performed with O5-CP1. qPCR reaction was performed using O5-p1 or O6-p2 in combination with CP1-p1, both on the ligation reaction with and without T4 DNA Ligase. Panel 10B shows technical replicates of qPCR reactions from the ligation reactions performed with O6-Cp1. qPCR reaction was performed using O5-p1 or O6-p2 in combination with CP1-p1, both on the ligation reactions with and without T4 DNA Ligase. In the O6 PCR+T4 DNA ligase the reaction curves came up in the expected order with the 64 fM and H₂O being indistinguishable from each other. Panel 10C shows technical replicates of qPCR reactions from the ligation reactions performed with the Universal1-CP1. qPCR reaction was performed using O5-p1 or O6-p2 in combination with CP1-p1, both on the ligation reaction with and without T4 DNA Ligase. The reaction curves came up in the expected order for both the O5 and O6 PCR, but concentrations below 8 pM were indistinguishable.

FIG. 11: In vivo qLNA-PCR: Detection of O13 with O13-CP1 in mouse brain and liver tissue. Two mice (no. 3 and no. 4) were injected IV with 950 nmols/kg of LNA-mix-pool1 while two control mice (no. 1 and no. 2) were injected with PBS as control. 7 days after injection mice were sacrificed and the small RNA fraction (<200 nt of length) from brain and liver tissue were purified and used as input in a LNA-DNA ligation using the Universal1-CP1. The ligation was used as input in a ddPCR reaction with the primers O13-p1 and CP1-p1. Panel 11A displays the raw fluorescence intensity in each droplet from the brain and liver samples of the 4 mice. The indicated threshold line was set manually and used to score the number of positive droplet. Panel 8B displays a bar chart of the number of positive droplets/events illustrating the clear differences seen between LNA oligonucleotides treated and untreated mice.

FIG. 12. Key design features of an exemplary Capture Probe for capturing, e.g. a sugar modified such as an LNA oligonucleotide, or a stereodefined oligonucleotide:

A: 5′ end is phosphorylated to enable ligation.
B: 3′ end is blocked for ligation to avoid self-ligation. A 3′ amino modification is illustrated but other 3′ blocking groups may be used.
C: Stretch of nucleotides base pairing to make intracellular loop, stabilizing the positioning the target LNA towards the 5′phosphate to enhance ligation. 6 complementary base pairs are shown—other complementary regions, as described herein as regions 1A and 2A may be used.
D: Internal hexa-ethyleneglycol-spacer. Flexible spacer allowing easy self baseparing and preventing read-through of polymerase. Other linker groups may be used as described herein.
E: An overhang free for base-pairing used to capture and bind the LNA-oligonucleotide temporarily to promote the double strand dependent ligation. The overhang can be sequence specific to capture a specific sequence or as illustrated here be comprised of 6 mixed basepairs enabling the capture of the oligonucleotide sequence. The length of the overhang can be varied as described herein, but is typically at least 2 or 3 nucleotides.

FIG. 13. qLNA-PCR: Schematic presentation of Reactions

FIG. 14. Generalized capture probe of the invention (annotations are mentioned in the claims): Arrows represent sequences of nucleosides orientated in the 5′→3′ direction.
Region 1A comprises at least 3 contiguous nucleotides; the 5′ terminal nucleotide is a DNA nucleotide which comprises a 5′ phosphate group (A).
Region 1B is an optional sequence of nucleotides which may comprise a predetermined sequence or a degenerate sequence.
Region 1C is a region of nucleotides which comprises a predetermined primer binding site (referred to as the universal primer site).
Region 2A is a region of nucleotides which are complementary to region 1A which form a duplex with region 1A (C).
Region 2B is a region of at least 2 or 3 nucleotides which form a 3′ overhang (E), the 3′ terminal nucleoside is blocked at the 3′ position (B) (i.e. does not comprise a 3′-OH group).
D is a linker moiety. In some embodiments D is a sequence of nucleotides. In some embodiments the linker moiety blocks DNA polymerase, such as a linker which comprises a non-nucleotide linker.

FIGS. 15 & 16. Generalized capture probes—as per FIG. 14 except that in (i) the linker moiety D is absent. The capture probe may therefore comprise two non-covalently linked oligonucleotide strands which hybridize between regions 1A and 2A (as shown in (i)). Note the 3′ ends are blocked (as illustrated by the star emblem). (ii), (iii) and (iv) show alternative oligonucleotide strands which may be ligated to the 3′ end of the nucleoside modified oligonucleotide, optionally in the presence of the first strand. Note that when the 2^{nd} strand is used in a ligation reaction to the 3′ terminus of a nucleoside modified oligonucleotide, region 1A need not have complementarity to region 2A, and may therefore be a sequence of nucleotides, wherein the 5′ most nucleotide is a DNA nucleoside. In some embodiments, region 1A may comprise the universal primer binding site (e.g. in (iv)). Alternatively an additional region 3′ to region 1A may be incorporated (1C) (iii) which incorporates the universal primer binding site, and region 1A may for example comprise a nested primer binding site, or a degenerate nucleotide sequence. Alternatively as illustrated in (ii) the nested primer binding site or a degenerate nucleotide sequence may be in region 1B.

FIG. 17: Determination of the effect of phosphorothioate chirality of a modified oligonucleotide as a T4 DNA ligase substrate. The figure illustrates that a Sp phosphorothioate internucleoside linkage between the two 3′ terminal nucleosides of a modified oligonucleotide do not provide an efficient substrate for T4DNA ligase, whereas the equivalent Rp phosphorothioate internucleoside linkage is an efficient substrate for T4DNA ligase.

FIG. 18: A mechanism of noise generation in the context of linker moiety “D” (see FIG. 15) being made of nucleic acid. During PCR or during optional step of reverse transcription it is likely that an LNA-specific primer will hybridize to the capture probe overhang (“2B”) and will get extended by the polymerase. This can be potentially avoided by designing the LNA-specific primer without complementarity to a “2B” region, but in most cases, due to usually utilized short LNA-oligonucleotides (13-20 nt), it is not possible. If a linker moiety “D” is made of nucleic acid (e.g. DNA) that can act as a DNA polymerase or reverse transcriptase template, then the extension will continue until the 5′ end of the capture probe, which is acting as a template and form a reverse complement of the capture probe with the LNA-specific primer at the 5′ end. Such a construct can fold back on itself (reverse complement of “1A”=“1A′” can hybridize with a reverse complement of “2A”-“2A′”) and the 3′ end can get extended forming a DNA product with an LNA-specific primer on its 5′ end and a complement of the LNA-specific primer on the 3′ end. Such a product can be PCR amplified with LNA-specific primer acting simultaneously as a forward and reverse primer and create background signal in the qPCR reaction, or non-LNA-oligonucleotide-derived bands in the gel based detection.

FIG. 19. An exemplary structure of a capture probe of the invention.

### DEFINITIONS

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. In the context of the present invention, oligonucleotides are man-made, and are chemically synthesized, and are typically purified or isolated.

### Nucleoside Modified Oligonucleotide

A nucleoside modified oligonucleotide is an oligonucleotide which comprises modified nucleosides, typically sugar modified nucleosides. In some embodiments the nucleoside modified oligonucleotide comprises at least one sugar modified nucleoside. In some embodiments the nucleoside modified oligonucleotide comprises at least one modified nucleoside at the 3′ end of the oligonucleotide, for example an LNA or a 2′substituted modified nucleoside, such as the at least two 3′ terminal nucleosides of the oligonucleotide are modified nucleosides, such as LNA or 2′ substituted modified nucleosides. In some embodiments the 3′ terminal nucleoside as a high affinity nucleoside analogue. In some embodiments the two 3′ terminal nucleosides are both high affinity nucleoside analogues. In some embodiments the two 3′ terminal nucleosides are both LNA nucleosides. In some embodiments the two 3′ terminal nucleosides are both 2′ substituted modified nucleosides, in particular 2′-O-MOE nucleosides. In some embodiments the oligonucleotide does not comprise a 5′ phosphate group.

### Capture Probe Oligonucleotide

A capture probe is an oligonucleotide which comprises at least one 5′ DNA nucleoside which is used to “capture” the nucleoside modified oligonucleotide. The capture may occur by the ligation of the 5′ end of the capture probe to the 3′ modified nucleotide of the modified nucleoside oligonucleotide. However, it is advantageous that the capture probe comprises a region which is complementary to a target nucleic acid sequence which is used to capture the target nucleic acid sequence via nucleic acid hybridization (Watson-Crick base pairing) prior to the ligation step. The invention provides optimized capture probes (e.g. as illustrated in FIG. 14) which may be used, but it will be recognized that for the methods and uses of the invention other capture probes may be used (See for examples the designs shown in FIGS. 15 & 16).

### Degenerate Nucleotides

A degenerate nucleotide refers to a position on a nucleic acid sequence that can have multiple alternative bases (as used in the IUPAC notation of nucleic acids) at a defined position. It should be recognized that for an individual molecule there will be a specific nucleotide at the defined position, but within the population of molecules in the oligonucleotide sample, the nucleotide at the defined position will be degenerate. In effect, the incorporation of the degenerate sequence results in the randomization of nucleotide sequence at the defined positions between each members of a population of oligonucleotides. In some embodiments, degenerate nucleotides (a degenerate nucleotide sequence) may be used in the capture probes of the invention to form the 3′ overhang (region 2C), for example in the event that the sequence of the oligonucleotide to be captured is not known or is not defined. Alternatively or in addition, a degenerate nucleotide sequence may be used down-stream of region 1A (e.g. optional region 1B) where it can, for example, act as a molecular “bar code” allowing the identification of unique ligation products.

### Predetermined Nucleotides

A predetermine nucleotide is a nucleotide that comprises a defined base (e.g. one of A, T, C or G) at a defined position within the oligonucleotide. A predetermined sequence is a sequence of predetermined nucleotides which has a known (designed) sequence. The capture probe of the invention comprises a predetermined sequence of nucleotides which form the universal primer binding site (1C) and the complementary regions 1A and 2A. Region 2B may also optionally comprise predetermined sequence, for example for use as a nested primer binding site or as a predetermined identifier sequence.

### Blocked 3′ Terminal Group

A blocked 3′ terminal group refers to a 3′ position on the 3′ terminal nucleoside of an oligonucleotide which does not comprise a —OH group. The blocked 3′ group does not therefore support enzymatic ligation (e.g. T4DNA ligase) or polymerase elongation (e.g. via Taq polymerse) from the 3′ end of the oligonucleotide. Numerous 3′ blocking groups are known in the art such as a nucleotidic modification which does not comprise a 3′-OH group, such as 3′deoxyribose, 2,3-dideoxyribose, 1,3-dideoxyribose, 1,2,3-trideoxyribose, and inverted ribose, a 3′ phosphate, 3′ amino, 3′ labels such as 3′ biotin, or a 3′fluorophore; or a non-nucleosidic modification, such as a non-ribose sugar, an abasic furan, a linker group (e.g. such as those described under region D herein), a thiol modifier (eg. C6SH, C3SH), an amino modifier, glycerol, or a conjugate group, such as fluorophores (fluorescein, AlexaFluor dyes, Atto dyes, cyanine dyes), digoxigenin, alkyne, azide, or cholesterol.

In some embodiments the 3′ blocking group is 3AmMO (3′ amino modification). In some embodiments the 3′ blocking group is a label, such as a fluorophore. In some embodiments the 3′ blocking group is not a fluorophore or is not a fluorescence quencher.

### Universal Primer/Universal Primer Binding Site

In the capture probe of the invention, region 1C comprises a universal primer binding site. This is a region of nucleotides with a predetermined nucleobase sequence which is used as a primer binding site (the Universal Primer) for first strand synthesis prior to PCR amplification: In the method of the invention, once the nucleoside modified oligonucleotide has been captured by the capture probe and the 5′ end of the capture probe has been ligated to the 3′ end of the sugar-modified oligonucleotide, a universal primer is hybridized to the universal primer binding site (region 1C) which is subsequently used for a DNA polymerase or reverse transcriptase mediated 5′-3′ chain elongation from the 3′ end of the universal primer across the length of the sugar-modified oligonucleotide, creating the first strand, or template molecule for PCR. A universal primer/universal primer binding site is typically at least 6 nucleotides in length (Ryu et al., Mol Biotechnol. 2000 January; 14(1):1-3), and may be for example 10-50 or 14-25 nucleotides in length.

In some embodiments the universal primer is a nucleotide primer, and may be a DNA primer or a modified DNA primer. In some embodiments the universal primer binding site is a region of nucleosides which are complementary to the universal primer, and may comprise DNA nucleotides and/or modified nucleotides.

### DNA Polymerase and Reverse Transcriptase

First strand synthesis may be performed using a DNA polymerase or a reverse transcriptase capable of reading the modified oligo nucleotide. In some embodiments, for use in PCR amplification on the first strand template, a thermostable DNA polymerase is used. Numerous DNA polymerases (also referred to herein as polymerases) are known in the art and may be employed for first strand synthesis and/or PCR, for example, in some embodiments the DNA polymerase is a thermostable polymerase such as a DNA polymerase selected from the group consisting of Taq polymerase, Hottub polymerase, Pwo polymerase, rTth polymerase, Tfl polymerase, Ultima polymerase, Volcano2G polymerase, and Vent polymerase.

The selection of the DNA polymerase/reverse transcriptase may be performed by evaluating the relative efficiency of the polymerase to read through the modified oligonucleotide, such as sugar-modified oligonucleotides. For sugar modified oligonucleotides, this may depend on the length of contiguous sugar-modified nucleosides in the oligonucleotide, and it is recognized that for heavily modified oligonucleotides an enzyme other than Taq polymerase may be desirable. The selection of the DNA polymerase/reverse transcriptase will also depend on the purity of the sample, it is well known that some polymerase enzymes are sensitive to contaminants, such as blood (See Al-Soud et al, Appl Environ Microbiol. 1998 October; 64(10): 3748-3753 for example).

In some embodiments the DNA polymerase is a Volcano2G DNA polymerase.

In some embodiments the first strand synthesis (elongation step) is performed using a reverse transcriptase. In some embodiments, the reverse transcriptase may be selected from the group consisting of M-MuLV Reverse Transcriptase, SuperScript™ III RT, AMV Reverse Transcriptase, Maxima H Minus Reverse Transcriptase.

### Blocks DNA Polymerase

A modification or linker moiety which blocks DNA polymerase prevents the read through of the polymerase across the linker moiety or modification, resulting in the termination of chain elongation.

### Polymerase Blocking Linker Moiety

The linker moiety of the capture probe of the invention is a moiety which links region 1C and region 2A, allowing the hybridization of the complementary nucleotides of regions 1A and 1C but preventing the polymerase (or reverse transcriptase) read-through across the linking moiety. The linker moiety may, in some embodiments, consist or comprise a non-nucleotide linker such as a non-nucleotide polymer, for example a alkyl linker, a polyethylene glycol linker, a non nucleosidic carbohydrate linker, a photocleavable linker (PC spacer), or an alkyl disulfide linker; or the linker moiety may consist or comprise a (poly) ribose based meoity, such as a region of 1,2-dideoxy ribose or abasic furan, or nucleosides which comprise non-hybridising base groups. It is recognized that some polymerases, such as some reverse transcriptases have sufficient promiscuity to jump across small regions of some linkers, and as such, the linker should be one which prevents read through of the polymerase to be used. For example, HIV reverse transcriptase can read through an abasic nucleoside, all be it with low efficiency (Cancio et al., Biochemical Journal 2004, 383(3) 475-482. Non-limiting examples of linker groups (D) are provided below

An alkyl spacer, e.g of structure

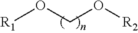

wherein n is at least 2, such as between 2-26, such as 2, 3, 6, 12, 18, 24, or 36. In some embodiments n=12
(http://www.linktech.co.uk/products/modifiers/spacer_modifiers/339_spacer-ce-phosphoramidite-c12)

In some embodiments n=3 C3 spacer (n=3)

http://www.linktech.co.uk/products/modifiers/spacer_modifiers/333_spacer-ce-phosphoramidite-c3

An ethyleneglycol based spacers, e.g of structure

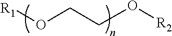

wherein n is at least 1, such as 2, 3, 4, 5, 6, 7, 8, 9, 10, 11 or 12. In some embodiments the linker is HEG (hexaethyleneglycol)
http://www.linktech.co.uk/products/modifiers/spacer_modifiers/337_spacer-ce-phosphoramidite-18-heg

In some embodiments the linker is a TEG (triethylene glycol) spacer.

http://www.linktech.co.uk/products/modifiers/spacer_modifiers/331_spacer-ce-phosphoramidite-9-teg
A Region of (Poly) 1,2-dideoxy ribose/Abasic Furan

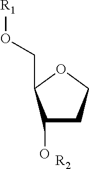

Wherein the region comprises at least one of said abasic furan such as 2, 3, 4, 5, 6, 7, 8, 9, 10 such abasic furan units, such as 6-50 abasic furan units.

### A C6 Disulfide Linker of Formula

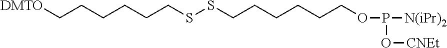

### A PC Linker Phosphoramidite of Formula:

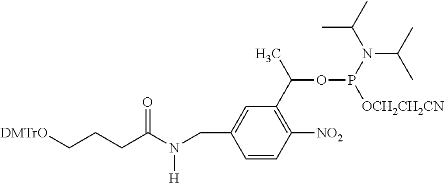

http://www.linktech.co.uk/products/modifiers/photocleavable_modifiers/352_pc-spacer-ce-phosphoramidite
or of formula

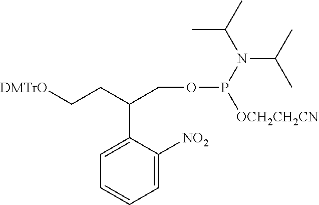

http://www.linktech.co.uk/products/modifiers/photocleavable_modifiers/354_pc-linker-ce-phosphoramidite

In some embodiments the linker moiety is a nucleotide based moiety but it comprises a modification which prevents polymerase read through, for example it may comprise an inverted nucleoside, or may comprise one or more modified nucleobases which do not allow (block) for hybridization.

### Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs. Typically antisense oligonucleotides comprise modified nucleosides, and are between 7-25 nucleotides in length, such as 7-20 nucleosides in length. Numerous designs of antisense oligonucleotides are known, several of which incorporate high affinity modified nucleosides, such as LNA, for example gapmer oligonucleotides, mixmer oligonucleotides etc. In some embodiments, the nucleoside modified oligonucleotide is an antisense oligonucleotide.

### Stereodefined Phosphorothioate Oligonucleotide

Typically, oligonucleotide phosphorothioates are synthesised as a random mixture of Rp and Sp phosphorothioate linkages (also referred to as a diastereomeric mixture).

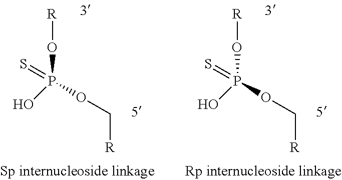

The above figure illustrates the stereochemistry of Sp and Rp phopshorothioate internucleoside linkages. R groups are nucleosides. Note the protonated form of the phosphorothaiote is shown for illustrative purposes only.

A stereodefined phosphorothioate oligonucleotide is a phosphorothioate oligonucleotide where at least one of the phosphorothioate linkages of the oligonucleotide is stereodefined, i.e. is either Rp or Sp in at least 75%, such as at least 80%, or at least 85%, or at least 90% or at least 95%, or at least 97%, such as at least 98%, such as at least 99%, or (essentially) all of the oligonucleotide molecules present in the oligonucleotide sample. Stereodefined oligonucleotides comprise at least one phosphorothioate linkage which is stereodefined. A fully stereodefined oligonucleotide is an oligonucleotide wherein all of the internucleoside linkages are stereodefined phosphorothioate internucleoside linkages. The term stereodefined, may be used to describe a defined chirality of one or more phosphorothioate internucleoside linkages as either Rp or Sp, or may be used to described a oligonucleotide which comprises such a (or more) phosphorothioate internucleoside linkage. It is recognised that a stereodefined oligonucleotide may comprise a small amount of the alternative stereoisomer at any one position, for example Wan et al reports a 98% stereoselectivity for the gapmers reported in NAR, November 2014.

The inventors have discovered that the capture probe and the methods of the invention may be used to detect, quantify, sequence, amplify, or clone a phosphorothioate oligonucleotide wherein the 3′ most internucleoside linkage of the phosphorothioate oligonucleotide is an Rp phosphorothioate internucleoside linkage.

As illustrated by the examples, the selective ability of T4DNA ligase to ligate an oligonucleotide with an Rp rather than an Sp phosphorothioate internucleoside linkage positioned between the two 3′ terminal nucleosides of a modified oligonucleotide can be used to discriminate between Rp and Sp internucleoside linkages. The method of the invention may therefore be used to identify the chirality of a phosphorothioate internucleoside linkage positioned between the two 3′ terminal nucleosides of an oligonucleotide.

### Detection of Other Oligonucleotides

It will be recognized that the method of the invention is not necessarily limited to the detection or quantification of antisense oligonucleotides, but may be employed in the detection or quantification or sequencing or cloning or other therapeutic oligonucleotides, such as siRNAs or aptamers, and may be used for the detection or quantification or sequencing or cloning of other nucleic acid sequences, e.g. microRNAs and cDNAs.

### Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a deoxyribose/ribose sugar moiety, a nucleobase moiety and one or more phosphate groups (when the phosphate group(s) is absent it is refered to as a nucleoside). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”.

### Modified Internucleoside Linkage

In some embodiments, the nucleoside modified oligonucleotide comprises internucleoside linkages other than phosphodiester—i.e. a “modified internucleoside linkage”. In some embodiments, the modified internucleoside linkage increases the nuclease resistance of the oligonucleotide compared to a phosphodiester linkage. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

In some embodiments the modified internucleoside linkages may be phosphorothioate internucleoside linkages. In some embodiments, the modified internucleoside linkages are compatible with the RNaseH recruitment of the oligonucleotide of the invention, for example phosphorothioate.

In some embodiments the internucleoside linkage comprises sulphur (S), such as a phosphorothioate internucleoside linkage.

A phosphorothioate internucleoside linkage is particularly useful due to nuclease resistance, beneficial pharmakokinetics and ease of manufacture. In some embodiments all of the internucleoside linkages of the oligonucleotide, or contiguous nucleotide sequence thereof, are phosphorothioate.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an oligonucleotide comprising one or more sugar-modified nucleosides and/or modified internucleoside linkages. In some embodiments the modified nucleoside comprises a stereodefined Rp phosphorothioate internucleoside linkage between the 2 3′ most (3′ terminal) nucleosides. In some embodiments, the modified oligonucleotide is a sugar-modified oligonucleotide. In some embodiments the sugar-modified oligonucleotide comprises a 3′ terminal nucleoside which is sugar-modified, such as a 2′ substituted nucleoside, such as 2′-O-MOE, or is a LNA nucleoside. In some embodiments, the sugar-modified oligonucleotide comprises a 3′ terminal LNA nucleoside, such as a beta-D-oxy LNA nucleoside or a (S) cET nucleoside.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex.

### High Affinity Modified Nucleosides

A high affinity modified nucleoside, also referred to as high affinity nucleoside analogues herein, is a modified nucleoside which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

### 2′ Modified Nucleosides.

A 2′ sugar modified nucleoside is a nucleoside which has a substituent other than H or —OH at the 2′ position (2′ substituted nucleoside) or comprises a 2′ linked biradicle, and includes 2′ substituted nucleosides and LNA (2′-4′ biradicle bridged) nucleosides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

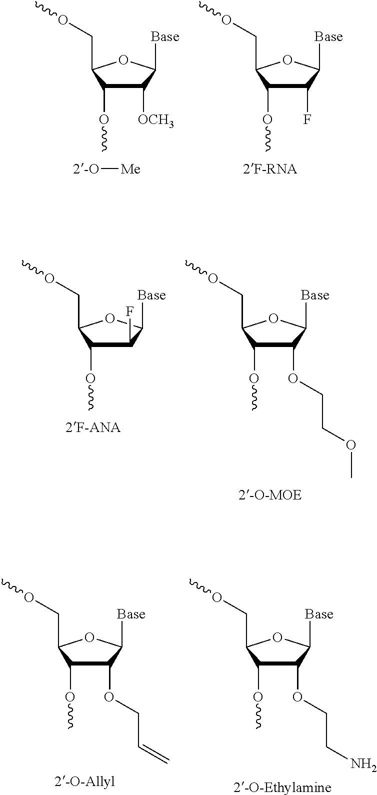

### Locked Nucleic Acid Nucleosides (LNA).

LNA nucleosides are modified nucleosides which comprise a linker group (referred to as a biradicle or a bridge) between C2′ and C4′ of the ribose sugar ring of a nucleotide. These nucleosides are also termed bridged nucleic acid or bicyclic nucleic acid (BNA) in the literature.

In some embodiments, the modified nucleoside or the LNA nucleosides of the oligomer of the invention has a general structure of the formula I or II:

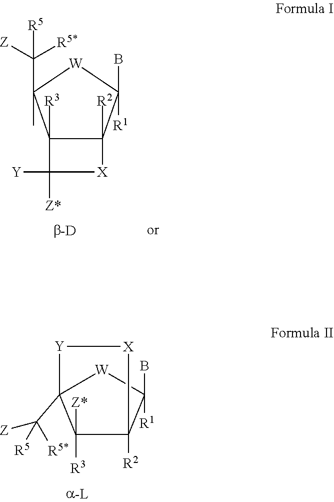

wherein W is selected from —O—, —S—, —N(R^{a})—, —C(R^{a}R^{b})—, such as, in some embodiments —O—;
B designates a nucleobase or modified nucleobase moiety;
Z designates an internucleoside linkage to an adjacent nucleoside, or a 5′-terminal group;
Z* designates an internucleoside linkage to an adjacent nucleoside, or a 3′-terminal group;
X designates a group selected from the list consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z

In some embodiments, X is selected from the group consisting of: —O—, —S—, NH—, NR^{a}R^{b}, —CH₂⁻, CR^{a}R^{b}, —C(═CH₂)—, and —C(═CR^{a}R^{b})—

In some embodiments, X is —O—

Y designates a group selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z

In some embodiments, Y is selected from the group consisting of: —CH₂—, —C(R^{a}R^{b})—, —CH₂CH₂—, —C(R^{a}R^{b})—C(R^{a}R^{b})—, —CH₂CH₂CH₂—, —C(R^{a}R^{b})C(R^{a}R^{b})C(R^{a}R^{b})—, —C(R^{a})═C(R^{b})—, and —C(R^{a})═N—

In some embodiments, Y is selected from the group consisting of: —CH₂—, —CHR^{a}—, —CHCH₃—, —CR^{a}R^{b}—

or —X—Y— together designate a bivalent linker group (also referred to as a radicle) together designate a bivalent linker group consisting of 1, 2, 3 or 4 groups/atoms selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z,

In some embodiments, —X—Y— designates a biradicle selected from the groups consisting of: —X—CH₂—, —X—CR^{a}R^{b}—, —X—CHR^{a}—, —X—C(HCH₃)⁻, —O—Y—, —O—CH₂—, —S—CH₂—, —O—CHCH₃—, —CH₂—O—CH₂, —O—CH(CH₃CH₃)—, —O—CH₂—CH₂—, OCH₂—CH₂—CH₂—, —O—CH₂OCH₂—, —O—NCH₂—, —O(═CH₂)—CH₂—, —NR^{a}—CH₂—, N—O—CH₂, —S—CR^{a}R^{b}— and —S—CHR^{a}—.

In some embodiments —X—Y— designates —O—CH₂— or —O—CH(CH₃)—.

wherein Z is selected from —O—, —S—, and —N(R^{a})—,
and R^{a} and, when present R^{b}, each is independently selected from hydrogen, optionally substituted C₁₋₆-alkyl, optionally substituted C₂₋₆-alkenyl, optionally substituted C₂₋₆-alkynyl, hydroxy, optionally substituted C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted and where two geminal substituents R^{a} and R^{b} together may designate optionally substituted methylene (═CH₂), wherein for all chiral centers, asymmetric groups may be found in either R or S orientation.
wherein R¹, R², R³, R⁵ and R^{5*} are independently selected from the group consisting of: hydrogen, optionally substituted C₁₋₆-alkyl, optionally substituted C₂₋₆-alkenyl, optionally substituted C₂₋₆-alkynyl, hydroxy, C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted, and where two geminal substituents together may designate oxo, thioxo, imino, or optionally substituted methylene.

In some embodiments R¹, R², R³, R⁵ and R^{5*} are independently selected from C₁₋₆ alkyl, such as methyl, and hydrogen.

In some embodiments R¹, R², R³, R⁵ and R^{5*} are all hydrogen.

In some embodiments R¹, R², R³, are all hydrogen, and either R⁵ and R^{5*} is also hydrogen and the other of R⁵ and R^{5*} is other than hydrogen, such as C₁₋₆ alkyl such as methyl.

In some embodiments, R^{a} is either hydrogen or methyl. In some embodiments, when present, R^{b} is either hydrogen or methyl.

In some embodiments, one or both of R^{a} and R^{b} is hydrogen

In some embodiments, one of R^{a} and R^{b} is hydrogen and the other is other than hydrogen

In some embodiments, one of R^{a} and R^{b} is methyl and the other is hydrogen

In some embodiments, both of R^{a} and R^{b} are methyl.

In some embodiments, the biradicle —X—Y— is —O—CH₂—, W is O, and all of R¹, R², R³, R⁵ and R^{5*} are all hydrogen. Such LNA nucleosides are disclosed in WO99/014226, WO00/66604, WO98/039352 and WO2004/046160 which are all hereby incorporated by reference, and include what are commonly known as beta-D-oxy LNA and alpha-L-oxy LNA nucleosides.

In some embodiments, the biradicle —X—Y— is —S—CH₂—, W is O, and all of R¹, R², R³, R⁵ and R^{5*} are all hydrogen. Such thio LNA nucleosides are disclosed in WO99/014226 and WO2004/046160 which are hereby incorporated by reference.

In some embodiments, the biradicle —X—Y— is —NH—CH₂—, W is O, and all of R¹, R², R³, R⁵ and R^{5*} are all hydrogen. Such amino LNA nucleosides are disclosed in WO99/014226 and WO2004/046160 which are hereby incorporated by reference.

In some embodiments, the biradicle —X—Y— is —O—CH₂—CH₂— or —O—CH₂—CH₂—CH₂—, W is O, and all of R¹, R², R³, R⁵ and R^{5*} are all hydrogen. Such LNA nucleosides are disclosed in WO00/047599 and Morita et al, Bioorganic & Med. Chem. Lett. 12 73-76, which are hereby incorporated by reference, and include what are commonly known as 2′-O-4′O-ethylene bridged nucleic acids (ENA).

In some embodiments, the biradicle —X—Y— is —O—CH₂—, W is O, and all of R¹, R², R³, and one of R⁵ and R^{5*} are hydrogen, and the other of R⁵ and R^{5*} is other than hydrogen such as C₁₋₆ alkyl, such as methyl. Such 5′ substituted LNA nucleosides are disclosed in WO2007/134181 which is hereby incorporated by reference.

In some embodiments, the biradicle —X—Y— is —O—CR^{a}R^{b}—, wherein one or both of R^{a} and R^{b} are other than hydrogen, such as methyl, W is O, and all of R¹, R², R³, and one of R⁵ and R^{5*} are hydrogen, and the other of R⁵ and R^{5*} is other than hydrogen such as C₁₋₆ alkyl, such as methyl. Such bis modified LNA nucleosides are disclosed in WO2010/077578 which is hereby incorporated by reference.

In some embodiments, the biradicle —X—Y— designate the bivalent linker group —O—CH(CH₂OCH₃)— (2′ O-methoxyethyl bicyclic nucleic acid—Seth at al., 2010, J. Org. Chem. Vol 75(5) pp. 1569-81). In some embodiments, the biradicle —X—Y— designate the bivalent linker group —O—CH(CH₂CH₃)— (2′O-ethyl bicyclic nucleic acid—Seth at al., 2010, J. Org. Chem. Vol 75(5) pp. 1569-81). In some embodiments, the biradicle —X—Y— is —O—CHR^{a}—, W is O, and all of R¹, R², R³, R⁵ and R^{5*} are all hydrogen. Such 6′ substituted LNA nucleosides are disclosed in WO10036698 and WO07090071 which are both hereby incorporated by reference.

In some embodiments, the biradicle —X—Y— is —O—CH(CH₂OCH₃)—, W is O, and all of R¹, R², R³, R⁵ and R^{5*} are all hydrogen. Such LNA nucleosides are also known as cyclic MOEs in the art (cMOE) and are disclosed in WO07090071.

In some embodiments, the biradicle —X—Y— designate the bivalent linker group —O—CH(CH₃)—.
in either the R- or S-configuration. In some embodiments, the biradicle —X—Y— together designate the bivalent linker group —O—CH₂—O—CH₂— (Seth at al., 2010, J. Org. Chem). In some embodiments, the biradicle —X—Y— is —O—CH(CH₃)—, W is O, and all of R¹, R², R³, R⁵ and R^{5*} are all hydrogen. Such 6′ methyl LNA nucleosides are also known as cET nucleosides in the art, and may be either (S)cET or (R)cET stereoisomers, as disclosed in WO07090071 (beta-D) and WO2010/036698 (alpha-L) which are both hereby incorporated by reference).

In some embodiments, the biradicle —X—Y— is —O—CR^{a}R^{b}—, wherein in neither R^{a} or R^{b} is hydrogen, W is O, and all of R¹, R², R³, R⁵ and R^{5*} are all hydrogen. In some embodiments, R^{a} and R^{b} are both methyl. Such 6′ di-substituted LNA nucleosides are disclosed in WO 2009006478 which is hereby incorporated by reference.

In some embodiments, the biradicle —X—Y— is —S—CHR^{a}—, W is O, and all of R¹, R², R³, R⁵ and R^{5*} are all hydrogen. Such 6′ substituted thio LNA nucleosides are disclosed in WO11156202 which is hereby incorporated by reference. In some 6′ substituted thio LNA embodiments R^{a} is methyl.

In some embodiments, the biradicle —X—Y— is —C(═CH₂)—C(R^{a}R^{b})—, such as —C(═CH₂)—CH₂—, or —C(═CH₂)—CH(CH₃)—W is O, and all of R¹, R², R³, R⁵ and R^{5*} are all hydrogen. Such vinyl carbo LNA nucleosides are disclosed in WO08154401 and WO09067647 which are both hereby incorporated by reference.

In some embodiments the biradicle —X—Y— is —N(—OR^{a})—, W is O, and all of R¹, R², R³, R⁵ and R^{5*} are all hydrogen. In some embodiments R^{a} is C₁₋₆ alkyl such as methyl. Such LNA nucleosides are also known as N substituted LNAs and are disclosed in WO2008/150729 which is hereby incorporated by reference. In some embodiments, the biradicle —X—Y— together designate the bivalent linker group —O—NR^{a}—CH₃— (Seth at al., 2010, J. Org. Chem). In some embodiments the biradicle —X—Y— is —N(R^{a})—, W is O, and all of R¹, R², R³, R⁵ and R^{5*} are all hydrogen. In some embodiments R^{a} is C₁₋₆ alkyl such as methyl.

In some embodiments, one or both of R⁵ and R^{5*} is hydrogen and, when substituted the other of R⁵ and R^{5*} is C₁₋₆ alkyl such as methyl. In such an embodiment, R¹, R², R³, may all be hydrogen, and the biradicle —X—Y— may be selected from —O—CH₂— or —O—C(HCR^{a})—, such as —O—C(HCH₃)—.

In some embodiments, the biradicle is —CR^{a}R^{b}—O—CR^{a}R^{b}—, such as CH₂—O—CH₂—, W is O and all of R¹, R², R³, R⁵ and R^{5*} are all hydrogen. In some embodiments R^{a} is C₁₋₆ alkyl such as methyl. Such LNA nucleosides are also known as conformationally restricted nucleotides (CRNs) and are disclosed in WO2013036868 which is hereby incorporated by reference.

In some embodiments, the biradicle is —O—CR^{a}R^{b}—O—CR^{a}R^{b}—, such as O—CH₂—O—CH₂—, W is O and all of R¹, R², R³, R⁵ and R^{5*} are all hydrogen. In some embodiments R^{a} is C₁₋₆ alkyl such as methyl. Such LNA nucleosides are also known as COC nucleotides and are disclosed in Mitsuoka et al., Nucleic Acids Research 2009 37(4), 1225-1238, which is hereby incorporated by reference.

It will be recognized than, unless specified, the LNA nucleosides may be in the beta-D or alpha-L stereoisoform.

Certain examples of LNA nucleosides are presented in Scheme 1.

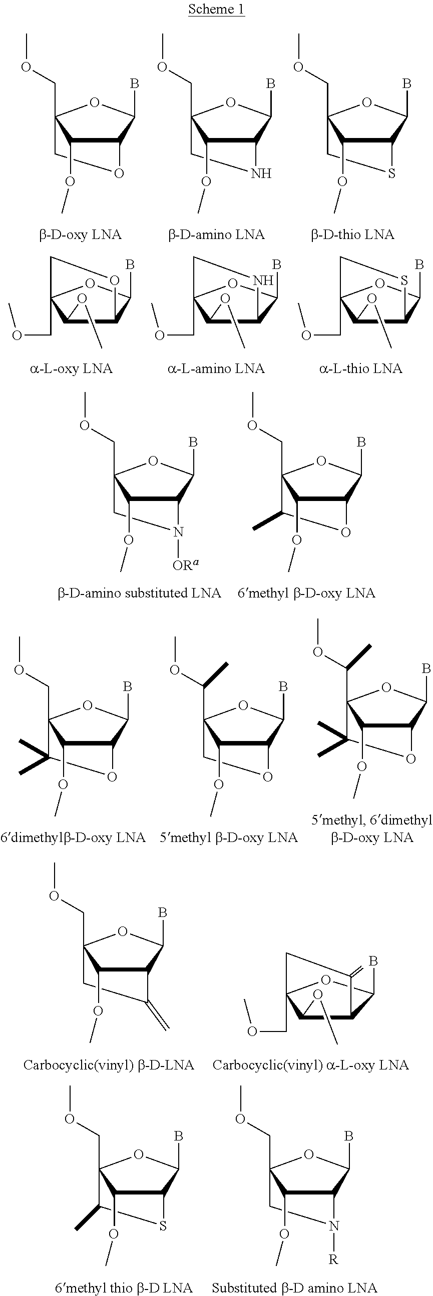

As illustrated in the examples, in some embodiments of the invention the LNA nucleosides in the oligonucleotides are beta-D-oxy-LNA nucleosides.

### 2′ Substituted Oligonucleotides

In some embodiments the nucleoside modified oligonucleotide comprises at least one 2′ substituted nucleoside, such as at least one 3′ terminal 2′ substituted nucleoside. In some embodiments the 2′ substituted oligonucleotide is a gapmer oligonucleotide, a mixmer oligonucleotide or a totalmer oligonucleotide. In some embodiments the 2′ substitution is selected from the group consisting of 2′methoxyethyl (2′-O-MOE) or 2′O-methyl. In some embodiments, the 3′ nucleotide of the nucleoside modified oligonucleotide is a 2′ substituted nucleoside such as 2′-O-MOE or 2′-O-methyl. In some embodiments the oligonucleotide does not comprise more than four consecutive nucleoside modified nucleosides. In some embodiments the oligonucleotide does not comprise more than three consecutive nucleoside modified nucleosides nucleosides. In some embodiments the oligonucleotide comprises 2 2′-O-MOE modified nucleotides at the 3′ terminal. In some embodiments the nucleoside modified oligonucleotide comprises phosphorothioate internucleoside linkages, and in some embodiments at least 75% of the internucleoside linkages present in the oligonucleotide are phosphorothioate internucleoside linkages. In some embodiments all of the internucleoside linkages of the modified nucleoside oligonucleotide are phosphorothioate internucleoside linkages. Phosphorotioate linked oligonucleotides are widely used for in vivo application in mammals, including their use as therapeutics.

In some embodiments the sugar modified oligonucleotide has a length of 7-30 nucleotides, such as 8-25 nucleotides. In some embodiments the length of the sugar modified oligonucleotide is 10-20 nucleotides, such as 12-18 nucleotides.

Nucleoside oligonucelotides may optionally be conjugated, e.g. with a GalNaC conjugate. If they are conjugated then it is preferable that the conjugate group is positioned other than at the 3′ position of the oligonucleotide, for example the conjugation may be at the 5′ terminal.

### LNA Oligonucleotide

In some embodiments the nucleoside modified oligonucleotide comprises at least one LNA nucleoside, such as at least one 3′ terminal LNA nucleoside. In some embodiments the LNA oligonucleotide is a gapmer oligonucleotide, a mixmer oligonucleotide or a totalmer oligonucleotide. In some embodiments the LNA oligonucleotide does not comprise more than four consecutive LNA nucleosides. In some embodiments the LNA oligonucleotide does not comprise more than three consecutive LNA nucleosides. In some embodiments the LNA oligonucleotide comprises 2 LNA nucleotides at the 3′ terminal.

### Gapmer

The nucleoside modified oligonucleotide may, in some embodiments be a gapmer oligonucleotide.

The term gapmer as used herein refers to an antisense oligonucleotide which comprises a region of RNase H recruiting oligonucleotides (gap—‘G’) which is flanked 5′ and 3′ by flanking regions (‘F’) which comprise one or more nucleoside modified nucleotides, such as affinity enhancing modified nucleosides (in the flanks or wings). Gapmers are typically 12-26 nucleotides in length and may, in some embodiments comprise a central region (G) of 6-14 DNA nucleosides, flanked either side by flanking regions F which comprises at least one nucleoside modified nucleotide such as 1-6 nucleoside modified nucleosides (F₁₋₆G₆₋14 F1-6). The nucleoside in each flank positioned adjacent to the gap region (e.g. DNA nucleoside region) is a nucleoside modified nucleotide, such as an LNA or 2′-O-MOE nucleoside. In some embodiments all the nucleosides in the flanking regions are nucleoside modified nucleosides, such as LNA and/or 2′-O-MOE nucleosides, however the flanks may comprise DNA nucleosides in addition to the nucleoside modified nucleosides, which, in some embodiments are not the terminal nucleosides.

### LNA Gapmer

The term LNA gapmer is a gapmer oligonucleotide wherein at least one of the affinity enhancing modified nucleosides in the flanks is an LNA nucleoside. In some embodiments, the nucleoside modified oligonucleotide is a LNA gapmer wherein the 3′ terminal nucleoside of the oligonucleotide is a LNA nucleoside. In some embodiments the 2 3′ most nucleosides of the oligonucleotide are LNA nucleosides. In some embodiments, both the 5′ and 3′ flanks of the LNA gapmer comprise LNA nucleosides, and in some embodiments the nucleoside modified oligonucleotide is a LNA oligonucleotide, such as a gapmer oligonucleotide, wherein all the nucleosides of the oligonucleotide are either LNA or DNA nucleosides.

### Mixed Wing Gapmer

The term mixed wing gapmer or mixed flank gapmer refers to a LNA gapmer wherein at least one of the flank regions comprise at least one LNA nucleoside and at least one non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA and 2′-F-ANA nucleoside(s). In some embodiments the mixed wing gapmer has one flank which comprises only LNA nucleosides (e.g. 5′ or 3′) and the other flank (3′ or 5′ respectfully) comprises 2′ substituted modified nucleoside(s) and optionally LNA nucleosides. In some embodiments the mixed wing gapmer comprises LNA and 2′-O-MOE nucleosides in the flanks.

### Mixmers

A mixmer is an oligonucleotide which comprises both nucleoside modified nucleosides and DNA nucleosides, wherein the oligonucleotides does not comprise more than 4 consecutive DNA nucleosides. Mixmer oligonucleotides are often used for non RNAseH mediated modulation of a nucleic acid target, for example for inhibition of a microRNA or for splice switching modulation or pre-mRNAs.

### Totalmer

A totalmer is a nucleoside modified oligonucleotide wherein all the nucleosides present in the oligonucleotide are nucleoside modified. The totalmer may comprise of only one type of nucleoside modification, for example may be a full 2′-O-MOE or fully 2′-O-methyl modified oligonucleotide, or a fully LNA modified oligonucleotide, or may comprise a mixture of different nucleoside modifications, for example a mixture of LNA and 2′-O-MOE nucleosides. In some embodiments the totoalmer may comprise one or two 3′ terminal LNA nucleosides.

### Tinys

A tiny oligonucleotide is an oligonucleotide 7-10 nucleotides in length wherein each of the nucleosides within the oligonucleotide is an LNA nucleoside. Tiny oligonucelotides are known to be particularly effective designs for targeting microRNAs.

### DETAILED DESCRIPTION OF THE INVENTION

The inventors have identified that T4DNA ligase is capable of ligating a 5′ phosphorylated DNA nucleoside to the 3′ termini of a nucleoside modified oligonucleotide. The invention provides for the use of T4DNA ligase to ligate the 3′ terminus of a nucleoside modified oligonucleotide to the 5′ terminus of a DNA oligonucleotide, wherein the 3′ nucleoside of the nucleoside modified oligonucleotide is a modified nucleoside, such as a LNA nucleoside. The DNA oligonucleotide comprises at least one terminal DNA nucleoside, and may comprise 2 or 3 contiguous 5′ DNA nucleosides. Designs of such DNA oligonucleotides are disclosed herein, for example as illustrated in FIGS. 14, 15 & 16.

The inventors have also identified that DNA polymerases, for example thermostable DNA polymerases such as Taq polymerase, and reverse transcriptases, can effectively use a nucleoside modified template for (e.g. first) strand synthesis. The invention therefore provides for the use of DNA polymerase or reverse transcriptase, for first strand synthesis of a complementary nucleic acid from a nucleoside modified oligonucleotide. As described herein this use may be combined with the use of T4DNA ligase. The invention therefore provides for a method for producing a complementary DNA (cDNA) molecule from a nucleoside modified oligonucleotide, said method comprising the step of ligating a DNA oligonucleotide adaptor (e.g. a capture probe) to the 3′ end of the nucleoside modified oligonucleotide, followed by the step of hybridizing a primer to the DNA oligonucleotide adaptor, and then performing 5′-3′ DNA polymerase, or reverse transcriptase, mediated elongation from the primer to produce a cDNA which comprises a nucleobase sequence which is complementary to the nucleoside modified oligonucleotide. The method may further comprise the subsequent step of performing PCR amplification of the cDNA. This method may be used for detection, quantification, amplification, sequencing or cloning of the nucleoside modified oligonucleotide. In some embodiments, the nucleoside modified oligonucleotide comprises at least one (such as 1, 2, 3, 4 or 5) 3′ terminal modified nucleosides, such as at least one (such as 1, 2, 3, 4 or 5) LNA or at least one (such as 1, 2, 3, 4 or 5) 2′ substituted nucleosides, such as 2′O-MOE. In some embodiments, the nucleoside modified oligonucleotide comprises at least one non terminal modified nucleosides, such as LNA or a 2′ substituted nucleoside, such as 2′-O-MOE.

The inventors have designed capture probe oligonucleotides which can be used to capture, detect, amplify, quantify or sequence oligonucleotides and polynucleotides, such as nucleoside modified oligonucleotides.

The invention provides for a capture probe oligonucleotide comprising 5′-3′:

i) a first nucleotide segment comprising
a. at least 3 5′ contiguous nucleotides of predetermined sequence (1A), wherein the 5′ most nucleotide is a DNA nucleotide with a terminal 5′ phosphate group.
b. optionally a region of degenerate or predetermined nucleotides, positioned 3′ of region 1A (1B)
c. a 3′ region which comprises a universal primer binding site (1C);

ii) a second nucleotide segment, comprising
a. a contiguous sequence of nucleotides which are complementary to the predetermined sequence 1A of the first segment (2A)
b. a region of at least 2 nucleotides, wherein the 3′ most nucleotide is a terminal nucleotide with a blocked 3′ terminal group wherein the first and second regions are covalently linked via a linker moiety.

See FIG. 14 as an example of a generalized capture probe of the invention.

### Region 1A

In some embodiments, region 1A comprises or consists of at least 3 contiguous nucleotides, of predetermined sequence, wherein the 5′ terminal nucleotide is a DNA nucleotide which comprises a 5′ phosphate group (A). The at least 3 contiguous nucleotides are complementary to and can hybridize to region 2A. In some embodiments the at least 3 contiguous nucleotides of region 1A are DNA nucleotides.

In some embodiments, region 1A comprises or consists of at least 3 contiguous nucleotides, such as 3-10 contiguous nucleotides, such as 3-10 DNA nucleotides.

### Region 1B

Region 1B is an optional sequence of nucleotides positioned 3′ of region 1A which may comprise a predetermined sequence or a degenerate sequence, or in some embodiments both a predetermined sequence part and a degenerate sequence part. The length of region B, when present may be modulated according to use. When a degenerate sequence is used it may allow the “molecular bar coding” of amplification products in subsequent sequencing steps, allowing for the determination of whether a particular amplification product is unique. This allows for comparative quantification of different oligonucleotides present in a heterogenous mixture of oligonucleotides. In some embodiments region 1B comprises 3-30 degenerate contiguous nucleotides, such as 3-30 degenerate contiguous DNA nucleotides.

It is known that some sequences may be preferentially amplified during PCR, and as such by counting the occurance of a genetic “barcode sequence”, originating from the degenerate sequence, you can determine the pre-amplification relative quantities (see e.g. Kielpinski & Vinter, NAR (2014) 42 (8): e70.

In some embodiments region 1B introduces a semi-degenerate sequence, which allows benefit of both a bar code sequence and a predetermined sequence. Additional benefit is a quality control of the barcode sequence (see e.g. Kielpinski et al., Methods in Enzymology (2015) vol. 558, pages 153-180). A semi-degenerate sequence has a selected semi-degenerate nucleobase at each position (based upon the Need a definition of semi-degenerate—add IUPAC codes, R, Y, S, W, K, M, B, D, H and V (See table 3).

In some embodiments region 1B has both degenerate sequence and predetermined sequence, or has both degenerate sequence and semi-degenerate sequence, or has both predetermined sequence and semi-degenerate sequence, or has degenerate sequence and predetermined sequence and semi-degenerate sequence.

If region B comprises a predetermined sequence it may for example provide an alternative, or nested, primer site, upstream of the universal primer site, the use of nested primer sites is a well-known tool for reducing non-specific binding during PCR amplification. In some embodiments region 1B comprises 3-30 predetermined contiguous nucleotides, such as 3-30 predetermined contiguous DNA nucleotides.

In some embodiments the capture probe does not comprise region 1B.

### Region 1C

Region 1C is a region of nucleotides which comprises a predetermined primer binding site (also referred to as the universal primer binding site herein).

### Region 2A

Region 2A is a region of nucleotides which are complementary to region 1A which form a duplex with region 1A (FIG. 14-C). It is beneficial if region 2A does not comprise RNA nucleosides which are complementary to region 1A, and it is also beneficial that the nucleoside present in region 2A which is complementary to and hybridizes to the 5′ terminal nucleoside of the capture probe (5′ nucleoside of region 1A) is a DNA nucleoside. This results in the formation of a DNA/DNA duplex when regions 1A and 2A hybridize. In some embodiments the two or three 3′ most nucleosides of region 2A are DNA nucleosides. In some embodiments all of the nucleosides of region 2A are DNA nucleosides. In some embodiments, region 2A comprises at least 3 contiguous nucleotides that are complementary to and can hybridize to region 1A. In some embodiments the at least 3 contiguous nucleotides of region 2A are DNA nucleotides.

In some embodiments, region 2A comprises or consists of 3-10 contiguous nucleotides, such as 3-10 DNA nucleotides. In some embodiments, the nucleotides of region 1A and region 2A are DNA nucleotides. The length and composition (e.g. G/C vs NT) of the complementary sequences 1A and 2A may be used to modulate the strength of hybridization, allowing for optimization of the capture probe. It is also recognized that introduction of mismatches within a complementary sequence can be used to decrease the hybridization strength (see WO2014110272 for example). In some embodiments region 1A and 2A do not form a contiguous complementary sequence, but due to partial complementarity in some embodiments regions 1A and 2A form a duplex when admixed with the sample. The 3′ most base pair of regions 1A and 2A should be a complementary base pair, and in some embodiments the two or three most base pairs of regions 1A and 2A are complementary base pairs. In some embodiments, these 3′ base pair(s) are DNA base pairs.

### Region 2B

Region 2B serves the purpose of hybridizing the capture probe oligonucleotide to the nucleoside modified oligonucleotide that is to be detected, captured, sequenced and/quantified.

Region 2B is a region of at least two or three nucleotides which form a 3′ overhang (E), when region 1A and 2A, of the complementary sequences thereof, are hybridized. The 3′ terminal nucleoside of region 2B is blocked at the 3′ position (FIG. 14-B) (i.e. does not comprise a 3′-OH group).

In some embodiments, region 2B has a length of at least 3 nucleotides. The optimal length of region 2B may depend, at least on the length of the oligonucleotide to be captured, and the present inventors have found that region 2B can function with an overlap of 2 nucleotides, for example when using an RNase treated sample, and preferably is at least 3 nucleotides.

In some embodiments, region 2B comprises a degenerate sequence, or a semi-degenerate sequence, which allows for the capture of oligonucleotides without prior knowledge of the oligonucleotide sequence. The capture of oligonucleotides without prior knowledge of their sequence is particularly useful in identifying specific oligonucleotides from a library of different oligonucleotide sequences which have a desired biodistribution, or for the identification of partial oligonucleotide degradation products. The probes and methods of the invention may also be applied to the capture and identification of aptamers.

In some embodiments, region 2B comprises a predetermined sequence, allowing for the capture of nucleoside modified oligonucleotides with a known sequence. The use of a predetermined capture region 2B allows for capture, detection and quantification of therapeutic oligonucleotides in vivo, for example for pre-clinical or clinical development or subsequently for determining local tissue or cellular concentration or exposure in patient derived material. The determination of compound concentration in patients can be important in optimizing the dosage of therapeutic oligonucleotides in patients.

### Non-Hybridizing Linker Moiety (D)

D is a linker moiety which blocks DNA polymerase, such as a linker which comprises a non-nucleotide linker. Region D allows for the capture probe regions 1A and 2A to hybridize. The advantage of preventing read-through of the DNA polymerase from region 1C to 2A is that it prevents the formation of an alternative template molecule. Such alternative template molecules result in mispriming of the primers specific to the nucleoside modified oligonucleotide on the 5′ region of the capture probe. (FIG. 18)

In some embodiments of the invention the linker moiety D may be a region of nucleotides which allow region 1A and 2A to hybridise. Such a linker moiety would typically act as a template for DNA polymerase activity during PCR amplification, and as such the presence of the capture probe with a contiguous nucleotide sequence between region 1B and 2A may result in the co-amplification of a competing template during the PCR cycles, a particular issue when there is a low copy number of the initial template in the PCR reaction (or a low concentration of nucleoside modified oligonucleotide in the (e.g. patient) sample). It is therefore, when region D is a region of nucleotides, the region comprises a modification which prevents the read through activity of the polymerase, thereby avoiding the production of competing template molecules during PCR amplification. Such modifications may include the use of one or more non-hybridisable base moiety within the nucleotides of region D, or the use of inverted nucleotides.

In some embodiments region D comprises a polymerase blocking linker, such as a C₆₋₃₂ polyethyleneglycol linker, such as a C18 polyethyleneglycol linker or an alkyl linker. Other non-limiting exemplary linker groups which may be used are disclosed herein.

### Capture Probe Oligonucleotide Designs

In some embodiments regions 1A and 2A form a duplex of 3-20 base pairs, such as 6-15 base pairs, such as 6, 7, 8, 9, 10, 11, 12, 13, 14 or 15 base pairs. In some embodiments regions 1A and 2A form a region of DNA base pairs. In some embodiments, regions 1A and 2A form a region of 9 DNA base pairs.

In some embodiments, region C is between 10-30 nucleotides, such as 11, 12, 13, 14, 15, 16, 17, 18, 19, 20, 21, 22, 23, 24, 25, 26, 27, 28, 29 or 30 nucleotides. In some embodiments, region C is design to avoid significant self-complementarity either within region C or within the capture probe—such significant self-complementarity may create an undesirable secondary structure of the capture probe when used in the sample. In some embodiments, region C may consist or comprise of DNA nucleosides.

In some embodiments, when present regions 1B consists or comprises at least 3 contiguous degenerate nucleosides, such as 3, 4, 5, 6, 7, 8, 9 or 10 contiguous degenerate nucleosides.

In some embodiments, region 2B consists or comprises at least 4 contiguous degenerate nucleosides, such as 4, 5, 6, 7, 8, 9, 10, 11 or 12 contiguous degenerate nucleosides.

In some embodiments, the nucleosides of regions 1A, 2A, 2B and C, and when present are DNA nucleosides.

### Uses of the Capture Probe Oligonucleotide

The capture probe oligonucleotide may be used in detecting, quantifying, sequencing, amplifying or cloning an oligonucleotide in a sample, such as nucleoside modified oligonucleotides or an oligonucleotide wherein the 3′ most internucleoside linkage is a Rp phosphorothioate internucleoside linkage. The capture probe of the invention may be used for detecting, quantifying, sequencing, amplifying or cloning an oligonucleotide which comprises 3′ terminal modified nucleoside(s), such as high affinity nucleoside analogues and/or 2′ modified nucleosides, such as LNA nucleosides. The capture probe of the invention may be used for detecting, quantifying, sequencing, amplifying or cloning an oligonucleotide wherein said oligonucleotide further comprises a Rp phosphorothioate internucleoside linkage between the two 3′ most nucleosides of the oligonucleotide. The capture probe of the invention may be used for detecting, quantifying, sequencing, amplifying or cloning an oligonucleotide which comprises 3′ terminal modified nucleoside(s), such as high affinity nucleoside analogues and/or 2′ modified nucleosides, such as LNA nucleosides, wherein said oligonucleotide further comprises a Rp phosphorothioate internucleoside linkage between the two 3′ most nucleosides of the oligonucleotide. As illustrated herein the capture probes of the invention may be used to effectively capture LNA oligonucleotides, allowing for the detecting, quantifying, sequencing or cloning. 2′ modified oligonucleotides such as 2′-O-MOE or LNA gapmers, mixmers, totalmers are being developed or have already been approved as therapeutic oligonucleotides. The capture probe of the invention may be used to detecting, quantifying, sequencing or cloning phosphorothioate modified internucleoside linkages.

In some embodiments, the use of the capture probe oligonucleotide of the invention is for detecting, amplifying or quantifying a nucleoside modified oligonucleotide in a biological sample, such as in a biopsy sample, a blood sample or a fraction thereof, such as blood serum or plasma sample.

In some embodiments, the use of the capture probe oligonucleotide of the invention is for detecting, amplifying cloning, or quantifying an oligonucleotide in a biological sample, such as in a biopsy sample, a blood sample or a fraction thereof, such as blood serum or plasma sample, wherein said oligonucleotide comprises a Rp phosphorothioate internucleoside linkage between the two 3′ most nucleosides of the oligonucleotide.

In some embodiments, the use is for sequencing or cloning the oligonucleotide. Whilst for many years oligonucleotide therapeutics has provided the promise of going from target sequence to drug designed by Watson-Crick base pairing rules, in practice this has been very difficult to achieve and it has recently become apparent that individual sequences of oligonucleotides may have a profound effect on the pharmacological distribution of an oligonucleotide. It is therefore difficult to presume that a compound which has been select on the basis of its outstanding effect in vitro will have the same outstanding effect in vivo—simply put, its biodistribution may result in accumulation in non-target tissues and a low pharmacological effect in the target tissue. The present invention provides for the first time a method of cloning and sequencing nucleoside modified nucleotides, allowing the identification of the cryptic sequences which result in the uptake in the desired tissues, and avoid accumulation in non-target tissues. This may be achieved by making libraries of oligonucleotides with different sequences (e.g. degenerate oligonucleotide libraries) and using them in a method for identifying a nucleoside modified oligonucleotide (sequence) which is enriched in a target tissue in a mammal, said method comprising:
a. Administering the mixture of nucleoside modified oligonucleotides with different nucleobase sequences to a mammal,
b. Allow for the oligonucleotides to be distributed within the mammal, for example for a period of at least 24-48 hours.
c. Isolate a population of modified oligonucleotides from the target tissue of the mammal,
d. Perform the oligonucleotide capture method according to the invention, including the step of sequencing the population of modified oligonucleotides, to
e. Identify nucleoside modified oligonucleotide sequences which are enriched in the target tissue of the mammal.

Alternatively, the method may be used to identify a nucleoside modified oligonucleotide (sequence) which has low accumulation in a non-target tissue in a mammal, said method comprising:
a. Administering the mixture of nucleoside modified oligonucleotides with different nucleobase sequences to a mammal,
b. Allow for the nucleoside modified oligonucleotides to be distributed within the mammal, for example for a period of at least 24-48 hours.
c. Isolate a population of modified oligonucleotides from the target tissue of the mammal, and
d. Perform the oligonucleotide capture method according to the invention, including the step of sequencing the population of modified oligonucleotides, to
e. Identify nucleoside modified oligonucleotide sequences which have a low accumulation in the non-target tissue of the mammal.

Typically, the step of isolation of the nucleoside modified oligonucleotides from the sample of tissue or cells obtained from the mammal (or patient) is RNase treated and may be further purified (e.g. via gel or column purification) prior to use in the oligonucleotide capture method of the invention.

The invention provides a method for detecting, quantifying, amplifying, sequencing or cloning a nucleoside modified oligonucleotide in a sample, said method comprising the steps of
a. Admixing a capture probe oligonucleotide and the sample,
b. Performing T4 DNA ligase mediated ligation of the 5′ terminus of the capture probe oligonucleotide and the 3′ terminus of the nucleoside modified oligonucleotide;
c. Add a universal primer which is complementary to the capture probe oligonucleotide,
d. Perform 5′-3′ chain elongation of the universal primer, and
e. Detect, quantify, sequence or clone the chain elongation product obtained in step d).

In the above method the optimized capture probe of the invention may be used: The invention provides a method for detecting, quantifying, amplifying, sequencing or cloning a nucleoside modified oligonucleotide in a sample, said method comprising the steps of
a. Admixing the capture probe oligonucleotide of the invention and the sample under conditions which allow hybridization of region 2B of the capture probe oligonucleotide to the nucleoside modified oligonucleotide,
b. Performing T4 DNA ligase mediated ligation of the 5′ terminus of the capture probe oligonucleotide and the 3′ terminus of the nucleoside modified oligonucleotide;
c. Add a universal primer which is complementary to region 1A of the capture probe oligonucleotide,
d. Perform 5′-3′ chain elongation of the universal primer, in the presence of (i.e. via) a DNA polymerase or reverse transcriptase, and
e. Detect, quantify, sequence or clone the chain elongation product obtained in step d).
   Methods for Detecting, Quantifying, Amplifying, Sequencing or Cloning Stereodefined Oligonucleotides which Comprise an Rp Phosphorothioate Internucleoside Linkage

The invention provides a method for detecting, quantifying, amplifying, sequencing or cloning an oligonucleotide in a sample, said method comprising the steps of
a. Admixing a capture probe oligonucleotide and the sample,
b. Performing T4 DNA ligase mediated ligation of the 5′ terminus of the capture probe oligonucleotide and the 3′ terminus of the nucleoside modified oligonucleotide;
c. Add a universal primer which is complementary to the capture probe oligonucleotide,
d. Perform 5′-3′ chain elongation of the universal primer, and
e. Detect, quantify, sequence or clone the chain elongation product obtained in step d).
   wherein said oligonucleotide comprises a Rp phosphorothioate internucleoside linkage between the two 3′ most nucleosides of the oligonucleotide

The invention provides a method for detecting, quantifying, amplifying, sequencing or cloning a nucleoside modified oligonucleotide in a sample, said method comprising the steps of
a. Admixing the capture probe oligonucleotide of the invention and the sample under conditions which allow hybridization of region 2B of the capture probe oligonucleotide to the nucleoside modified oligonucleotide,
b. Performing T4 DNA ligase mediated ligation of the 5′ terminus of the capture probe oligonucleotide and the 3′ terminus of the nucleoside modified oligonucleotide;
c. Add a universal primer which is complementary to region 1A of the capture probe oligonucleotide,
d. Perform 5′-3′ chain elongation of the universal primer, in the presence of (i.e. via) a DNA polymerase or reverse transcriptase, and
e. Detect, quantify, sequence or clone the chain elongation product obtained in step d),
   wherein said oligonucleotide comprises a Rp phosphorothioate internucleoside linkage between the two 3′ most nucleosides of the oligonucleotide.

### The Sample and Preparation Thereof

The sample may be a biological sample, such as a sample from an animal which has been administered the oligonucleotide.

In the methods disclosed herein, biological samples may be RNase and/or DNase treated prior to admixing with the oligonucleotide capture probe. Suitably RNase or DNase treatment is performed with enzymes which degrade RNA or DNA (respectively), but do not degrade nucleoside modified nucleotides or nucleotide modified nucleotides (e.g. phosphorothioates).

In the methods disclosed herein, biological samples may be RNase and/or DNase treated and an oligonucleotide fraction purified, e.g. via gel or column purification, prior to admixing with the oligonucleotide capture probe. In some embodiments, an oligonucleotide containing fraction is purified from a biological sample prior to admixing with the oligonucleotide capture probe. The sample referred to in the method(s) of the invention may therefore be an oligonucleotide enriched fraction obtained from the biological sample.

In the method(s) of the invention, prior to step a) an additional step of RNase and/or DNase treatment and/or purification of the sample may be performed. Furthermore, or alternatively, the ligation product of step b. may be purified prior to step c. Gel purification or column purification may be used for example after step b.

### PCR Amplification

In some embodiments, step e. of the method(s) of the invention comprises a PCR amplification of the chain elongation product. The PCR step may utilize a primer which comprises a region which is complementary to the nucleoside modified oligonucleotide or a part thereof. The PCR may be a qPCR (quantitative PCR) method, such as droplet digital PCR (ddPCR).

For the detecting, quantifying, sequencing, amplifying or cloning of oligonucleotides with an unknown sequence, it may be necessary to utilize a 3′ adaptor ligation strategy whereby a nucleotide adaptor of known sequence is ligated to the 3′ end of the first synthesized strand including the reverse complement sequence of the nucleoside modified oligonucleotide.

In some embodiments, the method comprises an additional step, performed after step d) said additional step comprises ligating a 3′ adaptor to the product obtained in step d, and performing PCR on the product obtained using a primer which is complementary to the 3′ adaptor and a primer which is complementary to the capture probe oligonucleotide, such as the universal primer [a primer complementary to region 1C].

In some embodiments, the method further comprises the step of cloning the PCR product obtained.

In some embodiments, the method further comprises the step of sequencing the PCR product obtained.

In some embodiments, the method is for detecting or quantifying a therapeutic nucleoside modified oligonucleotide in a patient sample.

### Other Applications

Security: DNA oligonucelotides with a unique sequence, for example have been developed to mark personal property or to contaminate thieves at the site of the theft. However, DNA oligonucleotides are inherently unstable in the environment, and as such the ability to detect the unique DNA oligonucleotides will deteriorate over time, and may be further accelerated by decontamination attempts. The use of nucleoside modified oligonucleotides in security and asset marking is therefore highly desirable as the modifications greatly enhance the stability of the oligonucleotides. The capture probe oligonucleotides of the present enable the detection of nucleoside modified oligonucleotides used in security and asset marking applications, and may be combined with PCR based detection methods.

### Further Embodiments Relating to Stereodefined Oligonucleotides

The invention provides for:
1. A capture probe oligonucleotide, for capture, detection, amplification or sequencing of an oligonucleotide which comprises a Rp phosphorothioate internucleoside linkage between the two 3′ terminal nucleosides on the oligonucleotide, comprising 5′-3′:
i) a first nucleotide segment comprising
   a. at least 3 5′ contiguous nucleotides of predetermined sequence (1A), wherein the 5′ most nucleotide is a DNA nucleotide with a terminal 5′ phosphate group.
   b. optionally a region of degenerate or predetermined nucleotides, positioned 3′ of region 1A (1B)
   c. a 3′ region which comprises a universal primer binding site [a predetermined region of nucleotides] (1C);
ii) a second nucleotide segment, comprising
   a. a contiguous sequence of nucleotides which are complementary to the predetermined sequence 1A of the first segment (2A)
   b. a region of at least 2 nucleotides, wherein the 3′ most nucleotide is a terminal nucleotide with a blocked 3′ terminal group
      wherein the first and second regions are covalently linked via a non-hybridizing linker moiety.
2. The capture probe oligonucleotide of embodiment 1, wherein the non-hybridizing linker is selected from the group consisting of an alkyl linker, a polyethylene glycol linker, a non nucleosidic carbohydrate linker, a photocleavable linker (PC spacer), a alkyl disulfide linker, a region of 1,2-dideoxy ribose or abasic furan, or a region of nucleosides which comprise non-hybridising base groups.
3. The capture probe oligonucleotide of embodiment 1 or 2, wherein region 1A comprises at least 2 or at least 3 contiguous DNA nucleotides.
4. The capture probe oligonucleotide of any one of embodiments 1-3, wherein the oligonucleotide comprises region 1B, wherein region 1B comprises a region of at least 3-30 degenerate nucleotides, such as DNA nucleotides.
5. The capture probe oligonucleotide of any one of embodiments 1-3, wherein the oligonucleotide comprises region 1B, wherein region 1B comprises a region of at least 3-30 predetermined nucleotides, such as DNA nucleotides.
6. The capture probe oligonucleotide of any one of embodiments 1-3, wherein the oligonucleotide does not comprises region 1B.
7. The capture probe oligonucleotide of any one of embodiments 1-6, wherein region 2A comprises DNA nucleotides which are complementary to region 1A.
8. The capture probe oligonucleotide of any one of embodiments 1-7, wherein region 2B comprises a region of at least 2 or 3 nucleotides which are complementary to the 3′ nucleotides of the nucleoside modified oligonucleotide.
9. The capture probe oligonucleotide according to any one of embodiments 1-8, wherein the first nucleotide segment comprises a further region 1D, positioned 3′ to region 1C . . . .
10. The capture probe oligonucleotide according to any one of embodiments 1-9, wherein the 3′ terminal on region 2B is selected from the group consisting of: a label,
11. Use of the capture probe oligonucleotide according to any one of embodiments 1-10, for use in detecting, quantifying, sequencing, amplifying or cloning an stereodefined oligonucleotide which comprises a Rp phosphorothioate internucleoside linkage between the two 3′ terminal nucleosides on the oligonucleotide.
12. The use according to embodiment 11, wherein said stereodefined oligonucleotide is a nucleoside modified oligonucleotide which comprises a 2′ sugar modified nucleoside, such as a 2′ substituted or a bicyclic nucleoside.
13. The use according to embodiment 11, wherein said nucleoside modified oligonucleotide comprises LNA nucleosides.
14. The use according to embodiment 13, wherein said nucleoside modified oligonucleotide is a LNA gapmer or an LNA mixmer.
15. The use according to any one of embodiments 11-14, wherein the stereodefined oligonucleotide is a fully stereodefined oligonucleotide.
16. The use according to any one of embodiments 11-15, wherein said use is for detecting or quantifying a stereodefined oligonucleotide in a biological sample, such as in a biopsy sample, a blood sample or a fraction thereof, such as blood serum or plasma, wherein the stereodefined oligonucleotide comprises a Rp phosphorothioate internucleoside linkage between the two 3′ terminal nucleosides of the oligonucleotide.
17. The use according to any one of embodiments 11-16, wherein said use is for sequencing or cloning the stereodefined oligonucleotide, wherein the stereodefined oligonucleotide comprises a Rp phosphorothioate internucleoside linkage between the two 3′ terminal nucleosides of the oligonucleotide.
18. The use according to any one of embodiments 11-17, wherein the steroedefined oligonucleotide comprises a 2′ sugar modified nucleoside, such as 2′ substituted or a LNA nucleoside as the 3′ terminal nucleoside.
19. A method for detecting, quantifying, sequencing, amplifying or cloning a stereodefined oligonucleotide in a sample, said method comprising the steps of
   a. Optionally perform an RNase and/or DNase treatment of the sample
   b. Admixing a capture probe oligonucleotide and the sample under conditions which allow hybridization of the capture probe oligonucleotide to the stereodefined oligonucleotide,
   c. Performing T4 DNA ligase mediated ligation of the 5′ terminus of the capture probe oligonucleotide and the 3′ terminus of the stereodefined oligonucleotide;
   d. Add a universal primer which is complementary to a of the capture probe oligonucleotide,
   e. Perform 5′-3′ chain elongation of the universal primer,
   f. Detect, quantify, sequence, amplify or clone the chain elongation product obtained in step e).
      wherein the stereodefined oligonucleotide comprises a Rp phosphorothioate internucleoside linkage between the two 3′ terminal nucleosides of the oligonucleotide
20. The method according to embodiment 19, wherein the capture probe oligonucleotide is as according to any one of embodiments 1-10, wherein in step b, region 2B of the capture probe oligonucleotide hybridizes to the 3′ region of the stereodefined oligonucleotide, and wherein the universal primer is complementary to region 1A of the capture probe oligonucleotide.
21. The method according to embodiment 19 or 20, wherein step f. comprises a PCR amplification of the chain elongation product.
22. The method according to embodiment 21, wherein said PCR step utilizes a primer which comprises a region which is complementary to the nucleoside modified oligonucleotide or a part thereof.
23. The method according to any one of embodiments 19-22, wherein said method comprises an additional step, performed after step e) said additional step comprises ligating a 3′ adaptor to the product obtained in step 2e.
24. The method of embodiment 23, wherein after the additional step, either (i) PCR is performed on the product obtained using a primer which is complementary to the 3′ adaptor and a primer which is complementary to the capture probe oligonucleotide of embodiments 1-10, such as the universal primer [a primer complementary to region 1C], and/or (ii) sequencing the product obtained.
25. The PCR method according to any one of embodiments 21-24, wherein the PCR is a qPCR.
26. The method according to embodiment 21-25, wherein the method further comprises the step of cloning the PCR product obtained.
27. The method according to embodiment 21-25, wherein the method further comprises the step of sequencing the PCR product obtained.
28. The method according to any one of embodiments 19-27, wherein said method is for detecting or quantifying a therapeutic stereodefined oligonucleotide in a patient sample, wherein the stereodefined oligonucleotide comprises a Rp phosphorothioate internucleoside linkage between the two 3′ terminal nucleosides of the oligonucleotide.
29. The use of T4DNA ligase to ligate the 3′ terminus of a stereodefined oligonucleotide to the 5′ terminus of a DNA oligonucleotide, wherein the wherein the stereodefined oligonucleotide comprises a Rp phosphorothioate internucleoside linkage between the two 3′ terminal nucleosides of the oligonucleotide.
30. The use according to embodiment 29, wherein the 3′ nucleoside of the stereodefined oligonucleotide is a modified nucleoside such as a LNA nucleoside or a 2′ substituted nucleoside, such as a 2-O-MOE nucleoside.

### Further Embodiments

1. A capture probe oligonucleotide, for use in PCR or sequencing of a sugar modified oligonucleotide, comprising 5′-3′:
i) a first nucleotide segment comprising
   a. at least 3 5′ contiguous nucleotides of predetermined sequence (1A), wherein the 5′ most nucleotide is a DNA nucleotide with a terminal 5′ phosphate group.
   b. optionally a region of degenerate or predetermined nucleotides, positioned 3′ of region 1A (1B)
   c. a 3′ region which comprises a universal primer binding site (1C);
ii) a second nucleotide segment, comprising
   a. a contiguous sequence of nucleotides which are complementary to the predetermined sequence 1A of the first segment (2A)
   b. a region of at least 2 nucleotides, wherein the 3′ most nucleotide is a terminal nucleotide with a blocked 3′ terminal group
   wherein the first and second regions are covalently linked via a non-hybridizing linker moiety.
2. The capture probe oligonucleotide of embodiment 1, wherein the non-hybridizing linker is selected from the group consisting of an alkyl linker, a polyethylene glycol linker, a non nucleosidic carbohydrate linker, a photocleavable linker (PC spacer), a alkyl disulfide linker, a region of 1,2-dideoxy ribose or abasic furan, or a region of nucleosides which comprise non-hybridising base groups.
3. The capture probe oligonucleotide of embodiment 1 or 2, wherein region 1A comprises at least 2 or at least 3 contiguous DNA nucleotides.
4. The capture probe oligonucleotide of any one of embodiments 1-3, wherein the oligonucleotide comprises region 1B, wherein region 1B comprises a region of at least 3-30 degenerate nucleotides, such as DNA nucleotides.
5. The capture probe oligonucleotide of any one of embodiments 1-3, wherein the oligonucleotide comprises region 1B, wherein region 1B comprises a region of at least 3-30 predetermined nucleotides, such as DNA nucleotides.
6. The capture probe oligonucleotide of any one of embodiments 1-3, wherein the oligonucleotide does not comprises region 1B.
7. The capture probe oligonucleotide of any one of embodiments 1-6, wherein region 2A comprises DNA nucleotides which are complementary to region 1A.
8. The capture probe oligonucleotide of any one of embodiments 1-7, wherein region 2B comprises a region of at least 2 or 3 nucleotides which are complementary to the 3′ nucleotides of the nucleoside modified oligonucleotide.
9. The capture probe oligonucleotide according to any one of embodiments 1-8, wherein the first nucleotide segment comprises a further region 1 D, positioned 3′ to region 1C. A region 1D could be used to provide the molecule with internal flexibility required to achieve self base pairing (2A-2B) in case when a short linker is chosen. It may also function as an outer primer site for setting up a nested PCR reaction. I could also be used to introduce a restriction site, which would be used to release the ligated product from the solid support if the 3′ end of the capture probe is immobilized. (This should properly be written in to lawyer langue).
10. The capture probe oligonucleotide according to any one of embodiments 1-9, wherein the 3′ terminal on region 2B is either a nucleotidic modification which does not comprise a 3′-OH group, such as a modification selected from the group consisting of 3′deoxyribose, 2′, 3′-dideoxyribose, 1′, 3′-dideoxyribose, 1′, 2′, 3′-trideoxyribose, an inverted ribose, a 3′ phosphate, 3′ amino, 3′ labels such as 3′ biotin, and a 3′fluorophore; or a non-nucleosidic modification, such as a non-nucleosidic modification selected from the group consisting of a non-ribose sugar, an abasic furan, a linker group (e.g. such as those according to embodiment 2), a thiol modifier (eg. C6SH, C3SH), an amino modifier, glycerol, or a conjugate, and a label.
11. Use of the capture probe oligonucleotide according to any one of embodiments 1-10, for use in detecting, quantifying, sequencing, amplifying or cloning a nucleoside modified oligonucleotide.
12. The use according to embodiment 11, wherein said nucleoside modified oligonucleotide comprises a 2′ sugar modified nucleoside, such as a 2′ substituted or a LNA nucleoside.
13. The use according to embodiments 12, wherein the nucleoside modified oligonucleotide comprises a 2′ sugar modified nucleoside, such as 2′ substituted or a LNA nucleoside as the 3′ terminal nucleoside.
14. The use according to any one of embodiments 11-13, wherein said nucleoside modified oligonucleotide comprises LNA nucleosides.
15. The use according to embodiment 14, wherein said nucleoside modified oligonucleotide is a LNA gapmer or an LNA mixmer.
16. The use according to any one of embodiments 11-15, wherein the nucleoside modified oligonucleotide further comprises phosphorothioate modified internucleoside linkages.
17. The use according to any one of embodiments 11-16, wherein the internucleoside linkage between the two 3′ terminal nucleosides of the nucleoside modified oligonucleotide is a stereodefined Rp phosphorothioate internucleoside linkage.
18. The use according to any one of embodiments 11-17, wherein said use is for detecting or quantifying a nucleoside modified oligonucleotide in a biological sample, such as in a biopsy sample, a blood sample or a fraction thereof, such as blood serum or plasma.
19. The use according to any one of embodiments 11-18, wherein said use is for sequencing, amplifying or cloning the nucleoside modified oligonucleotide.
20. A method for detecting, quantifying, sequencing, amplifying or cloning a nucleoside modified oligonucleotide in a sample, said method comprising the steps of
   a. Optionally perform an RNase treatment of the sample
   b. Admixing a capture probe oligonucleotide and the sample under conditions which allow hybridization of the capture probe oligonucleotide to the nucleoside modified oligonucleotide,
   c. Performing T4 DNA ligase mediated ligation of the 5′ terminus of the capture probe oligonucleotide and the 3′ terminus of the nucleoside modified oligonucleotide;
   d. Add a universal primer which is complementary to a of the capture probe oligonucleotide,
   e. Perform 5′-3′ chain elongation of the universal primer,
   f. Detect, quantify, sequence, amplify or clone the chain elongation product obtained in step e).
21. The method according to embodiment 20, wherein the capture probe oligonucleotide is as according to any one of embodiments 1-10, wherein in step b, region 2B of the capture probe oligonucleotide hybridizes to the 3′ region of the nucleoside modified oligonucleotide, and wherein the universal primer is complementary to region 1A of the capture probe oligonucleotide.
22. The method according to embodiment 21 or 21, wherein step f. comprises a PCR amplification of the chain elongation product.
23. The method according to embodiment 22, wherein said PCR step utilizes a primer which comprises a region which is complementary to the nucleoside modified oligonucleotide or a part thereof.
24. The method according to any one of embodiments 20-23, wherein said method comprises an additional step, performed after step e) said additional step comprises ligating a 3′ adaptor to the product obtained in step 2e, and performing either (i) PCR on the product obtained using a primer which is complementary to the 3′ adaptor and a primer which is complementary to the capture probe oligonucleotide of embodiments 1-10, such as the universal primer [a primer complementary to region 1C], and/or (ii) sequencing the product obtained.
25. The PCR method according to any one of embodiments 22-24, wherein the PCR is a qPCR.
26. The method according to embodiment 22-25, wherein the method further comprises the step of cloning the PCR product obtained.
27. The method according to embodiment 22-26, wherein the method further comprises the step of sequencing the PCR product obtained.
28. The method according to any one of embodiments 20-27, wherein said method is for detecting or quantifying a therapeutic nucleoside modified oligonucleotide in a patient sample.
29. A method for identifying a nucleoside modified oligonucleotide which is enriched in a target cell or tissue in a mammal, said method comprising:
   a. Administering a mixture of nucleoside modified oligonucleotides with different nucleobase sequences to a mammal,
   b. Allow for the oligonucleotides to be distributed within the mammal, for example for a period of at least 24-48 hours.
   c. Isolate a population of modified oligonucleotides from the target cell or tissue of the mammal,
   d. Perform the method according to any one of embodiments 20-28, including the step of sequencing the population of modified oligonucleotides, to
   e. Identify nucleoside modified oligonucleotide sequences which are enriched in the target tissue of the mammal.
30. The use of T4DNA ligase to ligate the 3′ terminus of a nucleoside modified oligonucleotide to the 5′ terminus of a DNA oligonucleotide, wherein the 3′ nucleoside of the nucleoside modified oligonucleotide is a LNA nucleoside.

### EXAMPLES

TABLE 1
Capture probe oligonucleotides and primers used
in the examples. All linkages are
phosphodiesters, all nucleotides are DNA
nucleotides unless preceded by “m” or “r”,
in which case they are 2′-O-methyl nucleotides
or ribonucleotides, respectively. “/5Phos/”
indicates 5′ phosphate group, “/36-FAM/”
indicates 3′ FAM group, /iSp18/ indicates
18-atom hexa-ethyleneglycol spacer /3AmMO/
indicates a 3′Amino modifier
(https://eu.idtdna.com/site/Catalog/
Modifications/Product/3299). Nucleotide codes
are as per IUPAC nucleotide code (see Table 3).

| Oligo- | | | SEQ |
|---|---|---|---|
| nucleotide | Oligonucleotide | | ID |
| ID | sequence | Type | NO |
| a1 | /5Phos/TGGAATTCTCGGGTGCC | Single | 1 |
| | AAGGA/36-FAM/ | Stranded | |
| | | Capture | |
| | | Probe | |
| a2 | /5Phos/NWTRYSNNNNTGGAATT | Single | 2 |
| | CTCGGGTGCCAAGGA/36-FAM/ | Stranded | |
| | | Capture | |
| | | Probe | |
| a3 | /5Phos/mUmGmGAATTCTCGGGT | Single | 3 |
| | GCCAAGGA/36-FAM/ | Stranded | |
| | | Capture | |
| | | Probe | |
| a4 | /5Phos/CTATCCAGCNNNTGGAA | A Capture | 4 |
| | TTCTCGGGTGCCAAGGA/iSp18/ | Probe of | |
| | GCTGGATAGNNNNNN/36-FAM/ | the | |
| | | invention | |
| a5 | /5Phos/rCrUrArUCCAGCNNNT | Capture | 5 |
| | GGAATTCTCGGGTGCCAAGGA/ | Probe | |
| | iSp18/GCTGGATAGNNNNNN/ | | |
| | 36-FAM/ | | |
| a6 | /5Phos/rCrUrArUCCAGCNNNT | Capture | 6 |
| | GGAATTCTCGGGTGCCAAGGA/ | Probe | |
| | iSp18/rGrCrUrGrGrArUrArG | | |
| | rNrNrNrNrNrN/36-FAM/ | | |
| a7 | /5Phos/CTATCCAGCNNNTGGAA | Capture | 7 |
| | TTCTCGGGTGCCAAGGA/iSp18/ | Probe | |
| | rGrCrUrGrGrArUrArGrNrNrN | | |
| | rNrNrN/36-FAM/ | | |
| O6-CP1 | /5Phos/ACGTCCAGCATCTGTTC | Capture | 8 |
| | AATTGGTACAAACTG/iSp18/GC | Probe | |
| | TGGACGTAATTCA/3AmMO/ | | |
| O5-CP1 | /5Phos/ACGTCCAGCATCTGTTC | Capture | 9 |
| | AATTGGTACAAACTG/iSp18/GC | Probe | |
| | TGGACGTGACAGT/3AmMO/ | | |
| Universal1- | /5Phos/ACGTCCAGCATCTGTTC | Capture | 10 |
| CP1 | AATTGGTACAAACTG/iSp18/GC | Probe | |
| | TGGACGTNNNNNN/3AmMO/ | | |
| O8-CP1 | /5Phos/CGGACCAGCAAGCTTAG | Capture | 11 |
| | AGATCACGGTATCCAGATTCGCTC | Probe | |
| | ATAGTACACAACTGCC/iSp18/T | | |
| | CCGACAGG/3AmMO/ | | |
| O6-p1 | TGTGCTATTCTGTGAATTAC | Forward | 12 |
| | | Primer | |
| O6-p2 | TGTGCTATTCTGTGAATT | Forward | 13 |
| | | Primer | |
| O5-p1 | GTTGACACTGTCACG | Forward | 14 |
| | | Primer | |
| CP1-p1 | CAGTTTGTACCAATTGAACA | Reverse | 15 |
| | | Primer | |
| O13-p1 | AGGAAAGCAAACAGTATATACGT | Forward | 16 |
| | | Primer | |

The a4 capture probe is illustrated in FIG. 19. Note the sequence listing refers to the DNA sequences of the above compound and does not reflect therefore the mixture of DNA and RNA nucleosides, or the non-nucleotide moieties presence within the above compounds.

TABLE 2
LNA containing oligonucleotides in this table
were synthesized with phosphorothioate linkages.
Uppercase characters indicate LNA, lowercase
characters indicate DNA. “/5Phos/” indicates 5′
phosphate group. N indicate a degenerate
nucleoside units

| Oligo- | | | |
|---|---|---|---|
| nucleotide | Oligonucleotide | | SEQ |
| ID | sequence | Type | ID NO |
| O1 | CTGATAAGCT | modified | 17 |
| | | oligo- | |
| | | nucleotide | |
| O2 | AGCattggtatTCA | modified | 18 |
| | | oligo- | |
| | | nucleotide | |
| O3 | CTCACtcggtcacga | modified | 19 |
| | GTG | oligo- | |
| | | nucleotide | |
| O4 | NNNnnnnnnnnnnNN | modified | 20 |
| | N | oligo- | |
| | | nucleotide | |
| O5 | GTtgacactgTC | modified | 21 |
| | | oligo- | |
| | | nucleotide | |
| O6 | TGTGctattctgtgA | modified | 22 |
| | ATT | oligo- | |
| | | nucleotide | |
| O7 | TTGaataagtggaTG | modified | 23 |
| | T | oligo- | |
| | | nucleotide | |
| O8 | GCaagcatcctGT | modified | 24 |
| | | oligo- | |
| | | nucleotide | |
| O9 | ACtcgtacttCCGA | modified | 25 |
| | | oligo- | |
| | | nucleotide | |
| O10 | AGTtataatccaGCT | modified | 26 |
| | | oligo- | |
| | | nucleotide | |
| O11 | TGTatcgactgcaTT | modified | 27 |
| | A | oligo- | |
| | | nucleotide | |
| O12 | CGTcagtatgcgAAT | modified | 28 |
| | c | oligo- | |
| | | nucleotide | |
| O13 | AGGaaagcaaacagt | modified | 29 |
| | aTAT | oligo- | |
| | | nucleotide | |
| O14 | Galnac- | modified | 30 |
| | C6GTtgacactgTC | oligo- | |
| | | nucleotide | |
| O15 | /5Phos/NNNnnnnn | modified | 31 |
| | nnnnnNNN | oligo- | |
| | | nucleotide | |

TABLE 3

| IUPAC nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

### Common Methodologies

### Oligonucleotide Mixture

Where a mix pool of LNA oligonucleotides (LNA-mix-pool1) was used this was prepared by mixing the following LNA oligonucleotides. (O5 10 μM, O6 5 μM, O7 10 μM, O8 10 μM, O9 10 μM, O10 10 μM, O11 10 μM, O12 10 μM, O13 10 μM, O14 10 μM). Oligonucleotide O6 was present in the mix in half the molar ratio of all the other LNA oligonucleotides. For ease of writing this mix pool will always be presented as an equimolar mix in the examples with the conc. being correct for all LNA oligonucleotides but O6 that always will be present in half the described concentration.

### LNA-DNA Ligation for PCR

All Ligation reactions before PCR reaction (Examples 7 to 10) were performed as follows: 2 μl LNA oligonucleotide containing sample was added to 2 μl capture probe oligonucleotide and mixed and incubated at 55° C. for 5 min. A mix containing 2 μl T4 DNA ligase (Thermo Scientific), 2 μl T4-DNA-ligase buffer, 8 μl PEG 4000 and 4 μl H₂O was added to each tube and mixed. The following program was run on a thermal cycler. 2 min 37° C., 3 min 30° C., 5 min 22° C., 30 min 16° C. this cycle was repeated twice, then 10 min at 70° C. and stable at 4° C.

Sybr Green qPCR:

Sybr Green qPCR was performed using the SYBR® Green SuperMix low Rox kit from Quantabio. All reactions were performed in 10 μL with the following setup: 5 μl SYBR® Green SuperMix, 100 nM forward primer, 100 nM reverse primer, 2 μl input template and H2O up to 10 μL.

SYBR Green PCR Program:

| | |
|---|---|
| Hot start: | 95° C. 5 min |
| 40x cycles of: | |
| Denaturation: | 95° C. 10 sek |
| Annealing/extention: | (variable) ° C. 30 sek |
| Melt curve: | |
| Denaturation | 95° C. 15 sek |
| Annealing | 60° C. 1 min |
| Detection of duplex melting | 60° C.->95° C. 0.05° C./s |

Droplet Digital PCR (ddPCR):

qLNA-PCR was performed with droplet digital PCR (emulsion PCR) using BioRad Automatic Droplet Generator (AutoDG) together with the OX200 droplet digital PCR system. The emulsion PCR was performed with QX200™ ddPCR™ EvaGreen Supermix and the Automated Droplet Generation Oil for EvaGreen. The PCR reaction that was used as input for the AutoDG was setup as follows: 11 μl ddPCR™ EvaGreen Supermix, forward primer (final conc. 100 nM), reverse primer (final conc. 100 nM), sample 2 μL and H₂O up to a total of 22 μL.

Following droplet generation the plate was sealed and run on the ddPCR program on a thermal cycler:

EvaGreen ddPCR Program:

| | |
|---|---|
| Hot start: | 95° C. 5 min |
| 40x cycles of: | |
| Denaturation: | 95° C. 30 sek |
| Anneling/extention: | (variable) ° C. 30 sek |
| Droplet stabilization: | 4° C. 5 min |
| | 90° C. 5 min |
| Hold: | 4° C. inf. |

Droplets were read on a QX200 droplet reader and the threshold was set manually.

### Example 1: An Attempt to Ligate Different Capture Probe Oligonucleotides with a Pool of LNA Containing Oligonucleotides

In the present example six different ligation reactions were performed in an attempt of ligating the LNA containing, phosphorothioated oligonucleotides with capture probe oligonucleotides. Two different enzymes were tested—CircLigase II and T4 RNA Ligase. These enzymes are known to allow ligation of single stranded DNA molecules or single stranded RNA molecules, respectively. Each of those enzymes was tested for three different capture probe oligonucleotide designs: (a1) DNA oligonucleotide with fixed sequence, (a2) DNA oligonucleotide with 10 nucleotides on the 5′ end partially randomized and (a3) modified a1 oligonucleotide, designed to carry 2′-O-methyl modification on three 5′ most nucleotides. Each of the capture probe oligonucleotides was modified with 5′ phosphate which was necessary for the ligase to perform the ligation and with 3′ FAM, which was necessary to block the 3′ hydroxyl group, which would otherwise act as an undesired substrate for the ligation reaction, as well as allowing for fluorescence based detection of the molecule. The ligation reactions were performed as follows:

Ligation with CircLigase 2:

A pool of nucleoside modified oligonucleotides o1, o2 and o3 (see table 2) was prepared to contain 10 μM concentration of each species. Prior to ligation, 1 μl of the pool was mixed with a selected capture probe oligonucleotide from table 1, either 1 μl of 100 μM a1, or 1 μl of 100 μM a2, or 1 μl of 100 μM a3, followed by incubation at 50° C. for 5 min and placing on ice.

In parallel, a master mix was prepared composed of 1.5 volumes of H₂O, 2 volumes of 50% PEG 4000, 0.5 volume of 50 mM MnCl₂, 1 volume of CircLigase II 10× Reaction Buffer (epicentre), 2 volumes of 5 M betaine and 1 volume of CircLigase II enzyme (epicentre).

Eight μl of the master mix was added to 2 μl of each of the prepared oligonucleotide-capture probe mixes followed by incubation for 3 hours at 60° C. followed by 10 min at 80° C. and held at 4° C. until analysis using gel electrophoresis as described below.

Ligation with T4 RNA Ligase:

A Pool of LNA oligonucleotides o1, o2 and o3 (see table 2) was prepared to contain 10 μM concentration of each species. Prior to ligation, 2 μl of the pool was mixed with a selected capture probe oligonucleotide from table 1, either 2 μl of 100 μM a1, or 2 μl of of 100 μM a2, or 2 μl of of 100 μM a3, followed by incubation in 50° C. for 5 min and placing on ice.

In parallel, a master mix was prepared composed of 8 volumes of 50% PEG 4000, 2 volumes of 10× T4 RNA Ligase buffer (Thermo Fisher Scientific), 2 volumes of 1 mg/ml BSA (Thermo Fisher Scientific), 2 volumes of 10 mM ATP and 2 volumes of 10 U/μl T4 RNA Ligase (Thermo Fisher Scientific, catalog number EL0021).

Sixteen μl of the master mix was added to 4 μl of each of the prepared oligonucleotide-capture probe mixes followed by incubation for 5 hours at 4° C. followed by 10 hours at 16° C. followed by 10 min at 70° C. and held at 4° C. until analysis using gel electrophoresis as described below.

Treatment without any Ligase:

Reaction was performed identically to “Ligation with T4 RNA Ligase” but with replacement of volume of 10 mM ATP and T4 RNA Ligase with H₂O.

### Gel Electrophoresis:

To each of the above mentioned reactions an equal volume of 2× Novex® TBE-Urea Sample Buffer (Thermo Fisher Scientific) was added and samples were heat denatured for 2 min at 95° C. and placed on ice. Ten μl of the prepared samples were loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System(Bio Rad) on a Blue Tray.

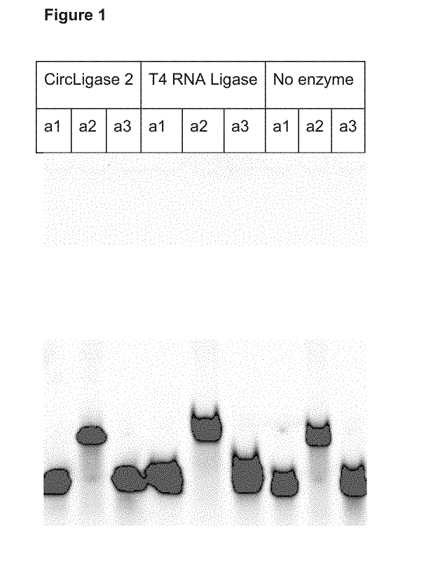

### Results (FIG. 1):

In the presented assay, the fluorescent signal after electrophoresis from samples treated with CircLigase II, T4 RNA Ligase or no ligase enzyme showed similar pattern and lacked the expected shift of the bands indicating ligation of the capture probe oligonucleotide to the LNA oligonucleotides. This indicates that within the detection limit of the utilized system there was no ligation between LNA oligonucleotides and tested capture probe oligonucleotides.

In conclusion none of the capture probe oligonucleotide-enzyme combinations yielded detectable ligation product.

### Example 2: Attempting Ligating of Different Capture Probe Oligonucleotides with a Pool of LNA Containing Oligonucleotides

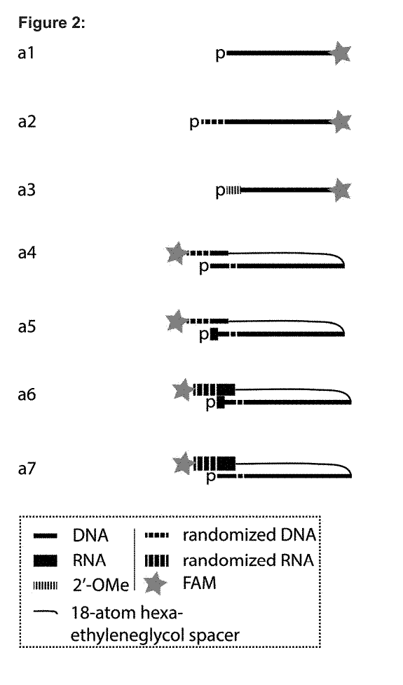

To overcome difficulties with ligation a capture probe oligonucleotide to an LNA-oligonucleotide, novel designs of the capture probe have been envisioned (FIG. 2 a4 to a7). These designs contain in addition to the nucleotide sequence to be attached to the LNA-oligonucleotide an also auxiliary overhang which is intended to partially hybridize to the capture probe oligonucleotide 5′ fragment as well as LNA-oligonucleotide 3′ fragment, forming a local double stranded structure.

In the present example we have tested multiple combinations of capture probe oligonucleotides (included in table 1) composed of DNA only (a4) or having four 5′-most nucleotides composed of RNA (the rest DNA) (a5) or having overhang composed of RNA (the rest DNA) (a6) or having both four 5′-most nucleotides composed of RNA and overhang composed of RNA (the rest DNA) (a7). An attempt of ligating these four different capture probe designs with four different ligase enzymes (T4 RNA Ligase, T4 DNA Ligase, T4 RNA Ligase 2, T7 DNA Ligase) was performed.

### Substrate Preparation:

Five and a half μl of 10 μM of LNA oligonucleotide o4 was mixed with 5.5 μl of 100 μM capture probe oligonucleotide a4, or a5 or a6 or a7, followed by incubation at 50° C. for 5 min and placing on ice.

### Master Mixes:

For Treatment without any enzyme, Master mix was prepared by combining 4 volumes of 50% PEG 4000, 3 volumes of H₂O and 1 volume of 10× T4 DNA Ligase Buffer (Thermo Fisher Scientific).

For treatment with T4 RNA Ligase, master mix was prepared by combining 4 volumes of 50% PEG 4000, 1 volume of 10× T4 RNA Ligase Buffer (Thermo Fisher Scientific), 1 volume of 1 mg/ml BSA (Thermo Fisher Scientific), 1 volume of 10 mM ATP and 1 volume of 10 U/μl T4 RNA Ligase (Thermo Fisher Scientific, catalog number EL0021).

For treatment with T4 DNA Ligase, master mix was prepared by combining 4 volumes of 50% PEG 4000, 1 volume of 10× T4 DNA Ligase Buffer (Thermo Fisher Scientific), 2 volumes of H2O and 1 volume of 30 U/μl T4 DNA Ligase HC (Thermo Fisher Scientific, catalog number EL0021).

For treatment with T4 RNA Ligase 2, master mix was prepared by combining 4 volumes of 50% PEG 4000, 1 volume of 10× T4 RNA Ligase 2 Buffer (New England Biolabs), 2 volumes of H2O and 1 volume of T4 RNA Ligase 2 (New England Biolabs, catalog number M0239S). For treatment with T7 DNA Ligase, master mix was prepared by combining 2 volumes of 50% PEG 4000, 5 volumes of 2× T7 DNA Ligase Buffer (New England Biolabs) and 1 volume of T7 DNA Ligase (New England Biolabs, catalog number M0318).

### Ligation Reaction:

Each master mix was split into 4 tubes, 8 μl to each. Two μl of the prepared substrate was added to each of the master mix, yielding in total 20 different combinations of enzyme and capture probe oligonucleotide. The mixture was incubated for (2 min at 37° C., 3 min at 30° C., 5 min at 22° C., 80 min at 16° C.)×2, hold at 4° C.

### Gel Electrophoresis:

To each of the above mentioned reactions an equal volume of 2× Novex® TBE-Urea Sample Buffer (Thermo Fisher Scientific) was added and samples were heat denatured for 2 min at 95° C. and placed on ice. Ten μl of thus prepared samples have been loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System(Bio Rad) on a Blue Tray.

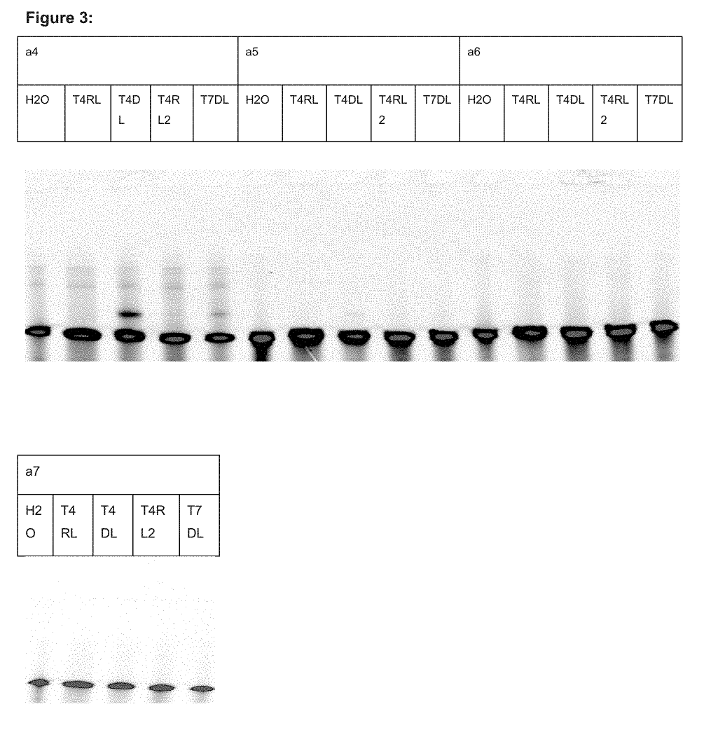

### Results (FIG. 3):

The most efficient ligation was observed for a combination of T4 DNA ligase and the capture probe oligonucleotide a4. Detectable ligation signal has also been obtained for reaction of T7 DNA Ligase with the capture probe oligonucleotide a4 and the reaction of T4 DNA Ligase with a5 capture probe oligonucleotide. No other reactions gave any detectable signal, indicating either lack of any reactivity or very low efficiency of the reaction. The results were compared with a control reaction devoid of any added ligase.

### Example 3: Exploring Ligation Conditions

In order to confirm that the observed band is indeed a product of ligation of the FAM labeled capture probe oligonucleotide and LNA oligonucleotide we have performed a series of reactions varying different parameters.

### Substrate Preparation:

For reaction “1”, 2 μl of 10 μM of LNA oligonucleotide o4 was mixed with 2 μl of 10 μM capture probe oligonucleotide a4; for reactions “2”, “4” and “5”, 2 μl of 10 μM of LNA oligonucleotide o4 was mixed with 2 μl of 100 μM capture probe oligonucleotide a4; for reaction “3”, 2 μl of 100 μM of LNA oligonucleotide o4 was mixed with 2 μl of 100 μM capture probe oligonucleotide a4. Mixing of LNA oligonucleotide with the capture probe oligonucleotide was followed by incubation at 50° C. for 5 min and placing on ice.

### Master Mixes:

Master mix for reactions “1”, “2” and “3” was prepared by combining 4 volumes of 50% PEG 4000, 1 volume of 10× T4 DNA Ligase Buffer (Thermo Fisher Scientific), 2 volumes of H₂O and 1 volume of 30 U/μl T4 DNA Ligase HC (Thermo Fisher Scientific, catalog number EL0021).

For reaction “4”, master mix was prepared in identical way as for reactions “1”, “2” and “3”, but it was heat treated by incubating at 70° C. for 10 min to inactivate the enzyme.

For reaction “5”, master mix was prepared in identical way as for reactions “1”, “2” and “3”, but T4 DNA Ligase HC has not been added, and its volume was replaced with H₂O.

### Ligation Reaction:

Ligation reactions were initiated by transferring 16 μl of the appropriate master mix to the prepared substrate and incubating for 5 h at 4° C., 10 h at 16° C., 10 min at 70° C. and kept at 4° C.

### Gel Electrophoresis:

To each of the above mentioned reactions an equal volume of 2× Novex® TBE-Urea Sample Buffer (Thermo Fisher Scientific) was added and samples were heat denatured for 2 min at 95° C. and placed on ice. Seven μl of thus prepared samples have been loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System(Bio Rad) on a Blue Tray.

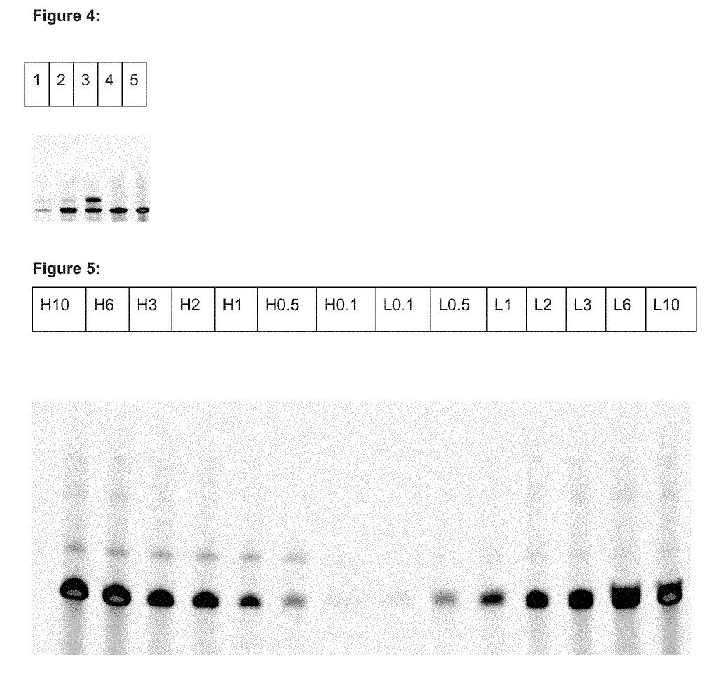

### Results (FIG. 4):

In reaction “1”, the amount of the capture probe oligonucleotide was reduced 10 times, yielding dramatic decrease of signal for unligated capture probe (lower band) and a slight decrease of signal for the ligated product (upper band). In reaction “3”, the amount of LNA oligonucleotide was increased 10 times, yielding dramatic increase of signal for ligated product, and slight decrease for unligated capture probe oligonucleotide. Reactions “4” and “5” were designed to investigate if the appearance of the ligated product is T4 DNA Ligase dependent, which is confirmed since the ligation product does not appear in the absence of the enzyme nor in the presence of heat inactivated enzyme.

### Example 4: Ligating a 5′ Phosphate Degenerate LNA Oligonucleotide (O15) with Capture Probe Oligonucleotide a4

In the present example LNA containing oligonucleotide “o15” was ligated to the capture probe oligonucleotide “a4” varying both o15 and a4 concentrations.

### Substrate Preparation:

One μl of 2 μM (“L”) or 10 μM (“H”) LNA oligonucleotide o15 was mixed with 1 μl of either 1, 5, 10, 20, 30, 60 or 100 μM capture probe oligonucleotide a4 and with 2 μl H2O, yielding 14 different combinations.

Mixing of LNA oligonucleotide with the capture probe oligonucleotide was followed by incubation at 50° C. for 5 min and placing on ice.

### Master Mix:

Master mix was prepared by combining 4 volumes of 50% PEG 4000, 1 volume of 10× T4 DNA Ligase Buffer (Thermo Fisher Scientific) and 1 volume of 30 U/μl T4 DNA Ligase HC (Thermo Fisher Scientific, catalog number EL0021).

### Ligation Reaction:

Ligation reactions were initiated by transferring 6 μl of the appropriate master mix to the prepared substrate and incubating for (2 min at 37° C., 3 min at 30° C., 5 min at 22° C., 80 min at 16° C.)×2, kept at 4° C.

### Gel Electrophoresis:

To each of the above mentioned reactions an equal volume of 2× Novex® TBE-Urea Sample Buffer (Thermo Fisher Scientific) was added and samples were heat denatured for 2 min at 95° C. and placed on ice. Seven μl of thus prepared samples have been loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System(Bio Rad) on a Blue Tray.

### Results (FIG. 5):

Quantification of the band with the ligated product revealed that the most efficient ligation for two tested concentration of o15 (final concentrations of 1 μM and 0.2 μM) occurred at the final concentration of a4 equal or higher than 2 μM.

### Example 5: Exploring Ligation Time

This experiment was designed to determine the minimal time needed for efficient ligation of the a4 capture probe oligonucleotide to a random pool of LNA oligonucleotides (o15).

Samples of two different concentrations of o15 were ligated for 0 up to 6 cycles, 20 minutes each.

### Substrate Preparation:

7.7 μl of 2 μM (“L”) or 10 μM (“H”) oligonucleotide o15 was mixed with 7.7 μl of 20 μM capture probe oligonucleotide a4 and with 15.4 μl H₂O.

Mixing of LNA oligonucleotide with the capture probe oligonucleotide oligonucleotide was followed by incubation at 50° C. for 5 min and placing on ice.

4 μl of the mix was removed and combined with 10 μl 2× Novex® TBE-Urea Sample Buffer (Thermo Fisher Scientific)—to act as samples L0 and H0.

### Master Mix:

Master mix was prepared by combining 4 volumes of 50% PEG 4000, 1 volume of 10× T4 DNA Ligase Buffer (Thermo Fisher Scientific) and 1 volume of 30 U/μl T4 DNA Ligase HC (Thermo Fisher Scientific, catalog number EL0021).

### Ligation Reaction:

Ligation reactions were initiated by transferring 40.2 μl of the appropriate master mix to the prepared substrate and incubating for (2 min at 37° C., 3 min at 30° C., 5 min at 22° C., 10 min at 16° C.) for 6 cycles, removing 10 μl after cycles 1, 2, 3, 4, 5 or 6 and combining with 10 μl 2× Novex® TBE-Urea Sample Buffer (Thermo Fisher Scientific) to stop the reaction. L0 and H0 samples, which already contained 10 μl 2× Novex® TBE-Urea Sample Buffer (Thermo Fisher Scientific) were combined with 6 μl of the master mix.

### Gel Electrophoresis:

To each of the above mentioned reactions an equal volume of 2× Novex® TBE-Urea Sample Buffer (Thermo Fisher Scientific) was added and samples were heat denatured for 2 min at 95° C. and placed on ice. Seven μl of thus prepared samples have been loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System(Bio Rad) on a Blue Tray.

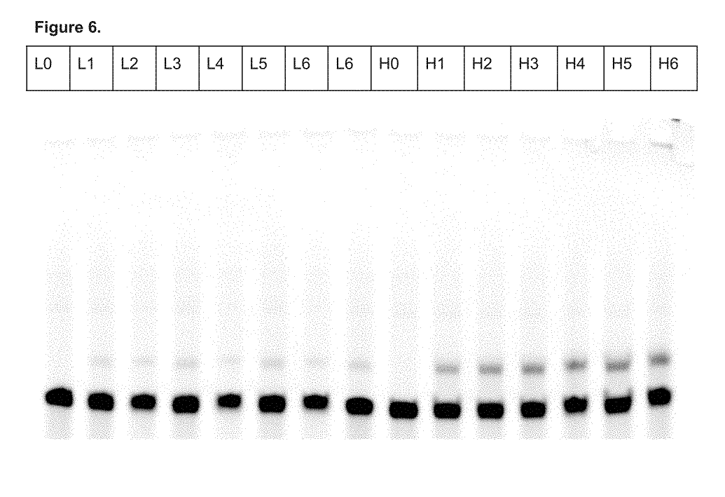

### Results (FIG. 6):

As the electrophoretic analysis of the ligated samples indicates, the majority of the ligation occurs within the first cycle with only modest improvements in subsequent cycles.

### Example 6: Exploring Optimal PEG 4000 Concentration

This experiment was designed to determine the optimal PEG 4000 concentration in the capture probe oligonucleotide ligation reaction.

### Materials and Methods

### Substrate Preparation:

5.5 μl of 2 μM (“L”) or 10 μM (“H”) LNA oligonucleotide o15 was mixed with 5.5 μl of 20 μM capture probe oligonucleotide a4 and with 11 μl H₂O.

Mixing of LNA oligonucleotide with the capture probe oligonucleotide oligonucleotide was followed by incubation at 50° C. for 5 min and placing on ice. Mixture was split into 5 tubes, 4 μl to each.

### Master Mix Preparation:

Master mix was prepared by combining 4 volumes of 0% or 10% or 20% or 30% or 40% or 50% PEG 4000, 1 volume of 10× T4 DNA Ligase Buffer (Thermo Fisher Scientific) and 1 volume of 30 U/μl T4 DNA Ligase HC (Thermo Fisher Scientific, catalog number EL0021).

### Ligation Reaction:

Ligation reactions were initiated by transferring 6 μl of each of the master mixes to the prepared substrate and incubating for (2 min at 37° C., 3 min at 30° C., 5 min at 22° C., 10 min at 16° C.) for 4 cycles, followed by adding 10 μl Novex® TBE-Urea Sample Buffer (Thermo Fisher Scientific) to stop the reaction.

L0 and H0 samples, which already contained 10 μl 2× Novex® TBE-Urea Sample Buffer (Thermo Fisher Scientific) were combined with 6 μl of the master mix.

### Gel Electrophoresis:

To each of the above mentioned reactions an equal volume of 2× Novex® TBE-Urea Sample Buffer (Thermo Fisher Scientific) was added and samples were heat denatured for 2 min at 95° C. and placed on ice. Seven μl of thus prepared samples have been loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System(Bio Rad) on a Blue Tray.

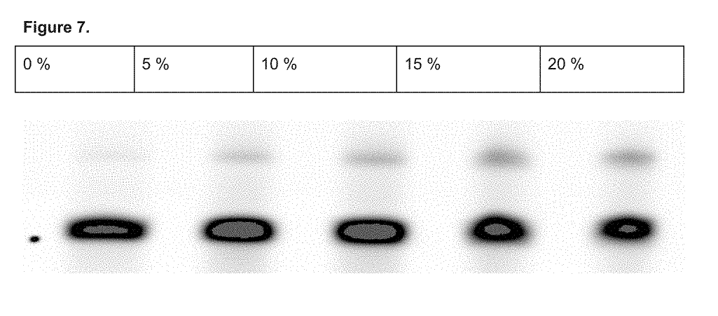

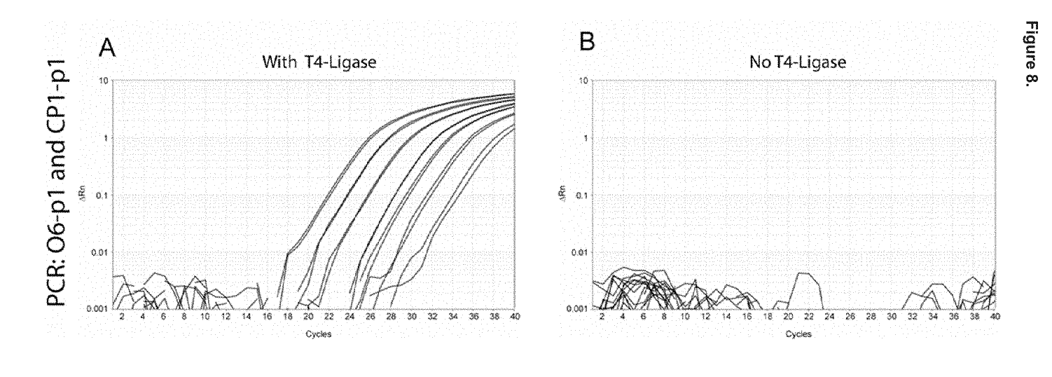

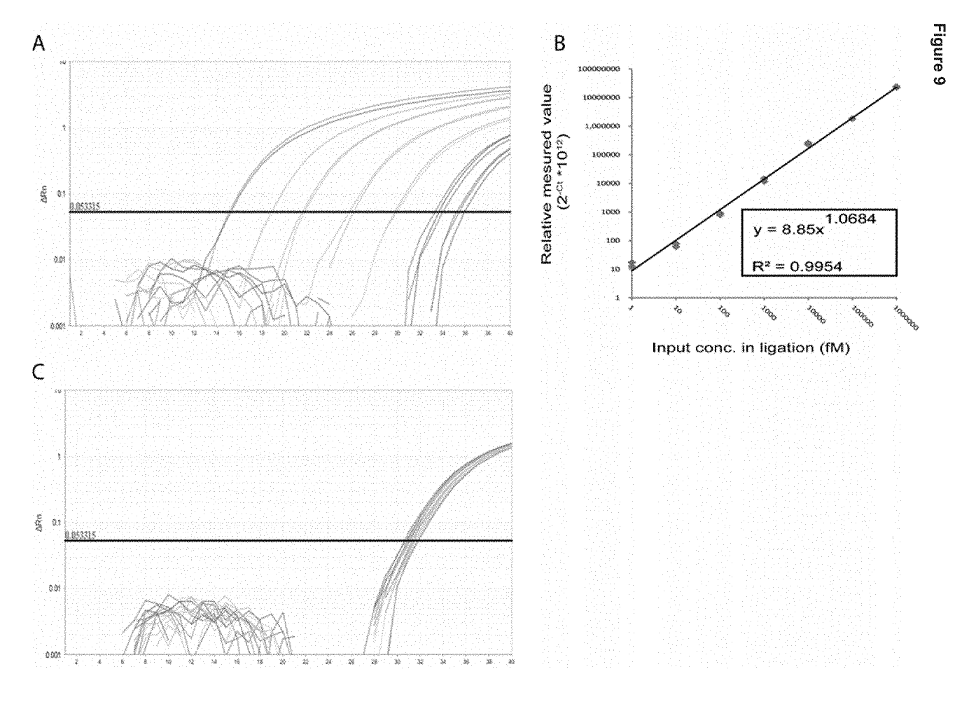

### Results (FIG. 7):

Quantification of the bands in the electrophoregram indicate that the most efficient ligation occurred with the final PEG 4000 concentration of 15%, closely followed by 20%.

### Example 7: Performing a PCR Reaction on the Product of the Ligation Between a LNA and a Capture Probe

To illustrate that a PCR reaction can be performed on the product of the ligation between a LNA oligonucleotide and a capture probe, we setup up a ligation reaction using LNA oligonucleotide 6(O6) and O6-capture probe 1 (O6-CP1). This reaction was done at high concentration in equimolar ratio. 100 μM O6 and 100 μM O6-CP1. The ligation was performed as described in the Materials and Methods section “LNA-DNA ligation for PCR” and was setup both in the absence and presence of T4-DNA Ligase. A dilution series of the ligation mix was made (250 pM, 62.5 pM, 15.6 pM, 3.9 pM, 1 pM, 244 fM, 61 fM) and used as input in a Sybr Green PCR reaction using the PerfeCTa SYBR® Green SuperMix kit from Quantabio that utilizes a modified Taq DNA polymerase (as described in the Materials and Method section “Sybr Green qPCR”). The ligated product was detected using a primer set consisting of the O6-p1 and CP1-p1 (see table 1 at an annealing temperature of 60° C. FIG. 8A displays the real-time PCR curves on the dilution series of the ligated product in the presence of T4 DNA Ligase. The different PCR products came up in the expected order with the 250 μM input reaction appearing first. FIG. 8B displays the same reactions except a T4-DNA-ligase was not present during ligation. We conclude that it is possible to ligate a LNA oligonucleotide with a capture probe oligonucleotide containing LNA and that the modified Taq polymerase was able to generate a PCR product from this ligated template molecule using the described primers.

### Example 8: Quantification of LNA Modified Oligonucleotides Using PCR Amplification

In order to use this new technique as a quantitative LNA detection method, it has to have a linear relation between LNA oligonucleotide input in the ligation and measured output in the PCR. We showed that this reaction is linear by setting up the experiment depicted in FIG. 9. In short, we created a pool comprised of 10 different LNA oligonucleotides (LNA-pool-1, see the Materials and Method Section). A 10× serial dilution of this LNA-pool-1 was made (1 nM, 100 μM, 10 μM, 1 μM, 100 fM, 10 fM, 1 fM, H20) and used as input material in a ligation reaction with capture probe oligonucleotide O6-CP1. This ligation reaction was set up in the absence or presence of T4-DNA-Ligase. All ligation reactions were diluted 9× before 2 μL were used as input in a 10 μl Sybr Green PCR reaction (see materials and methods section) using the primers O6-p2 and CP1-p1 using an annealing temperature of 52° C. FIG. 9A display the real-time PCR curves from the ligation reactions containing T4-DNA-Ligase. The PCR curves came up in the expected order for all the LNA oligonucleotide inputs, but the water sample also produced a product with a Ct value between the Ct value of the 10 fM and 1 fM reaction, illustrating that an unspecific reaction had occurred in this sample. FIG. 9B depicts a plot where the measured Ct value (2^{-Ct}*10¹²) were plotted against the concentration of LNA oligonucleotide input amount in the ligation reaction. This illustrates that the ligation reaction and PCR reaction follows nice linear correlation (R²=0.9954) and that the efficiency of the PCR reaction is close to 100%. (y=8.85x¹⁰⁶⁸⁴=>106.84% effectivity). FIG. 9C displays the PCR reaction on the ligation reaction were T4-DNA-ligase was not added. This illustrates that the PCR product originate from the ligated product, but also that a PCR by-product occurs independently of the presence of the LNA-DNA ligated product.

We conclude that this PCR based LNA oligonucleotide detection method is linear and can therefore be used as a quantitative method to measure LNA oligonucleotide concentrations. In the following the combined method of LNA-DNA ligation followed by a quantitative PCR reaction is termed quantitative LNA-PCR (qLNA-PCR).

### Example 9: qLNA-PCR Specificity

To examine the specificity of the qLNA-PCR method we set up an experiment to illustrate that the ligation of the capture probe oligonucleotide to a LNA oligonucleotide is sequence specific. In short, we made a 5× serial dilution of the LNA-pool-1 (1 nM, 200 pM, 40 pM, 8 pM, 1.6 pM, 320 fM, 64 fM, H20) and used it as input material in a ligation reaction with primers O5-CP1, O6-CP1 or Universal1-CP1 all done with and with-out presence of T4 DNA Ligase. Following a 9× dilution of the ligation reactions, 2 μL product of all ligations was used as input for two Sybr Green PCR reactions. A PCR using the primers O5-p1 and CP1-p1 with a 53° C. annealing temperature (O5 PCR) and a PCR using the primers O6-p2 and CP1-p1 with a 50° C. annealing temperature (O6 PCR) was conducted.

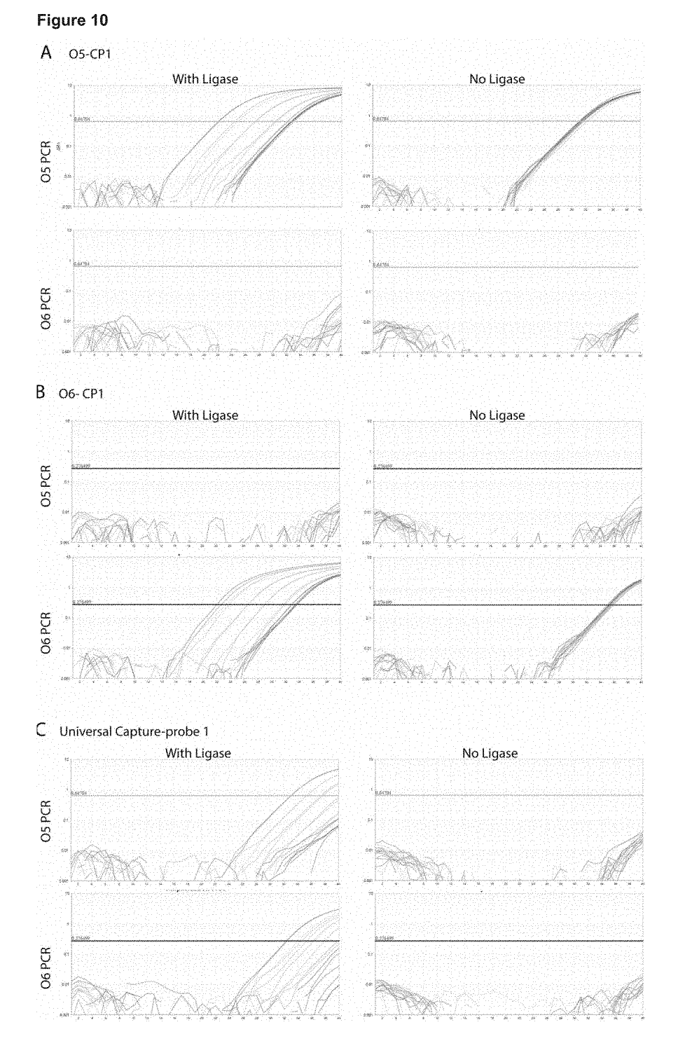

FIG. 10A displays the PCR reactions on the O5-CP1 ligations for both the O5 and O6 PCR with and without T4 DNA ligase presence during ligation. This illustrated that only the O5 PCR reaction occurred, meaning that O5-Cp1 did not ligate to the O6 LNA oligonucleotide. In the O5 PCR+T4 DNA ligase the reaction curves came up in the expected order with the 320 fM, 64 fM and H₂O being indistinguishable from each other, illustrating that an unspecific PCR reaction occurred which is also apparent from the O5 PCR on the product from the ligation reaction where T4 DNA ligase wasn't present.

FIG. 10B displays the PCR reactions from the O6-CP1 ligations for both the O5 and O6 PCR with and without T4 DNA ligase presence during ligation. This illustrates that only the O6 PCR reaction occurs, meaning that O6-Cp1 did not ligate to the O5 LNA oligoncleotide. Collectively this showed that these qLNA-PCR reactions were specific due to the sequence specific ligation of the LNA oligonucleotide and the DNA nucleosides in the capture probe oligonucleotide. In the O6 PCR+T4 DNA ligase the reaction curves came up in the expected order with the 64 fM and H₂O being indistinguishable from each other, again illustrating an unspecific PCR products was generated.

FIG. 100 shows that when the overhang extension of the capture probe oligonucleotide was replaced by a degenerated sequence (NNNNNN) the capture probe could capture both O5 and O6 LNA oligonucleotides, and hence the Universal1-CP1 can be used to detect and quantify any LNA oligooligonucleotide sequence, as long as a specific PCR reaction can occur on the product.

Collectively we concluded that qLNA-PCR can be very specific with specificity originating from both the ligation step and from the PCR step where a LNA specific primer is used.

### Example 10: In Vivo Detection of LNA Oligonucleotides

To illustrate that qLNA-PCR can be used in an in vivo setting to detect and quantify LNA oligonucleotides an in vivo mouse experiment was setup.

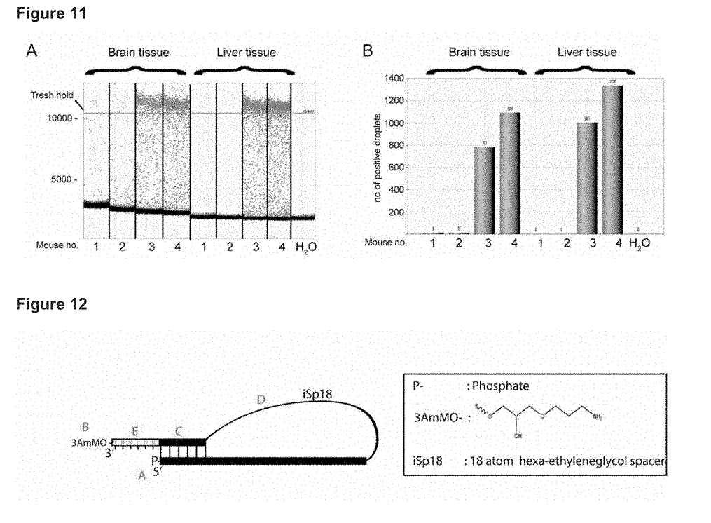

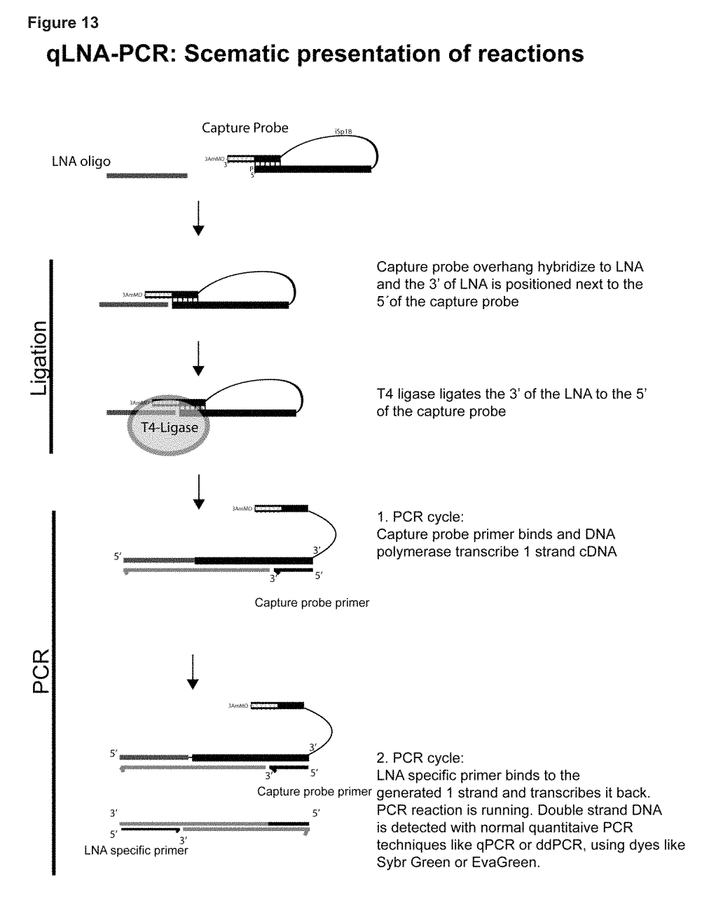

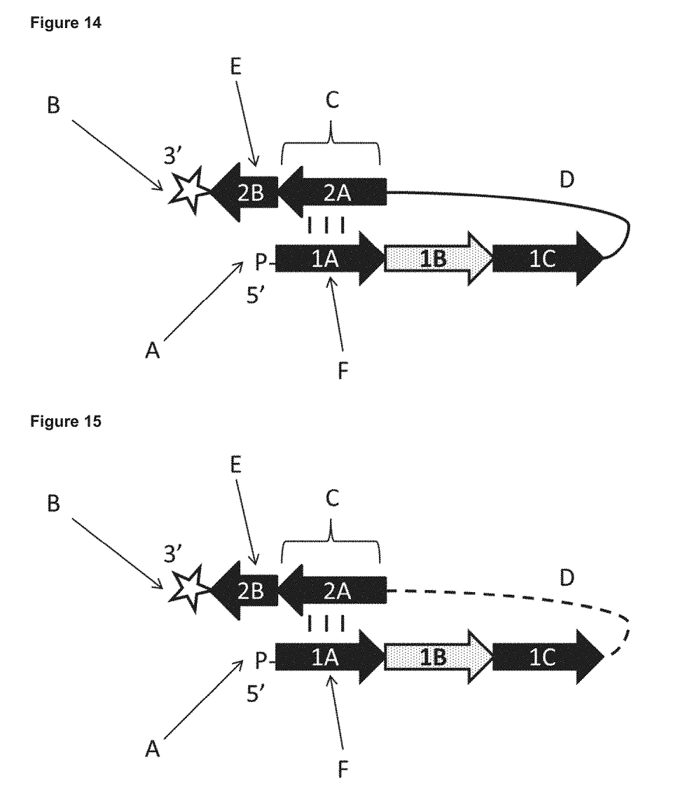

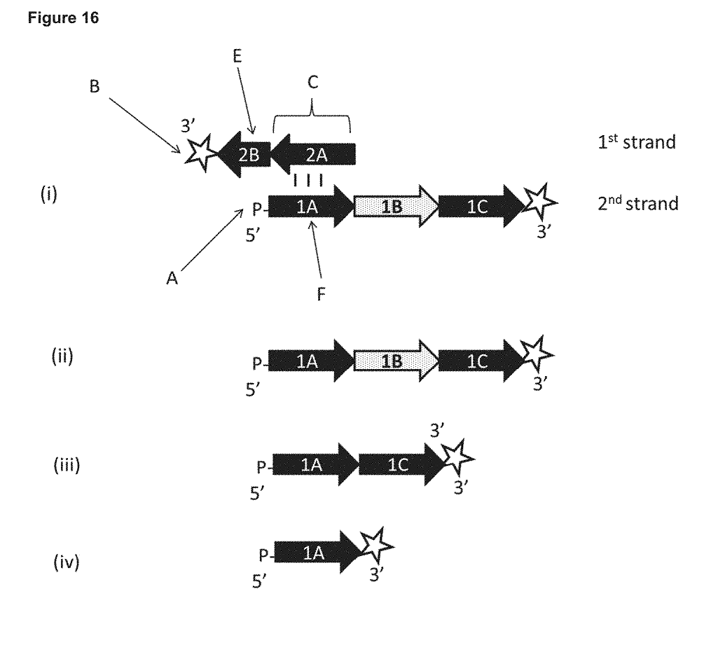

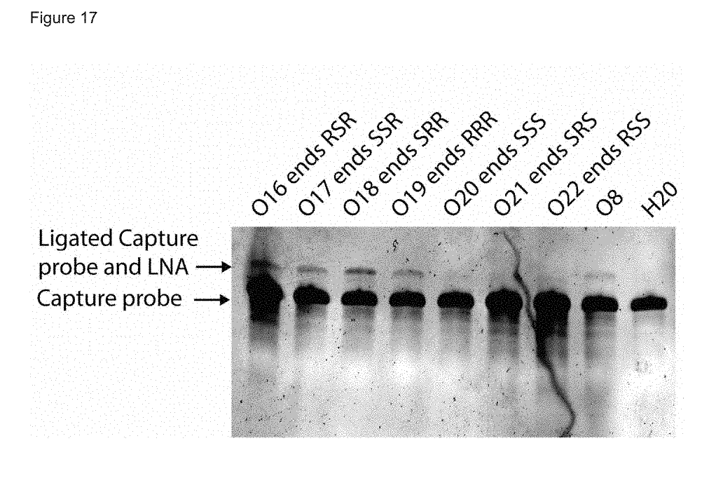

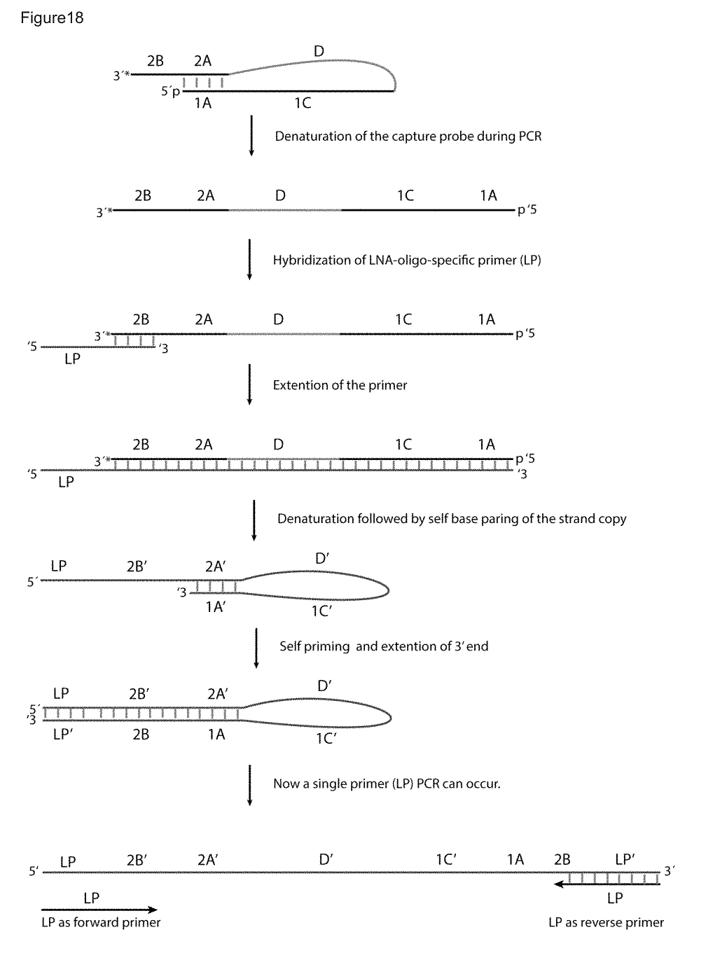

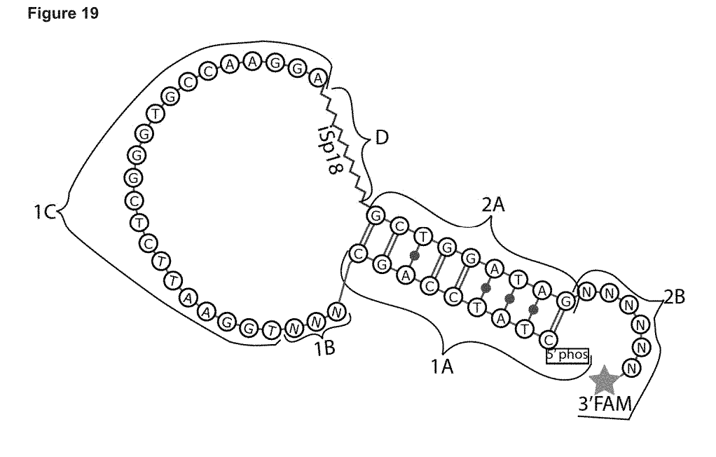

LNA-mix-pool1 was injected intravenously in two adult female C57 black with 950 nmol/kg of LNA oligonucleotide in total (100 nmol/kg of each LNA oligonucleotide, and 50 nmol/kg of oligonucleotide o6). Two control mice were injected with PBS. Seven days after injection the mice were sacrificed and brain and liver tissue was isolated and snap frozen with dry ice. The small RNA fraction <200 bp was isolated from 50 mg of brain tissue and 25 mg of liver tissue from each mouse using the miRNeasy kit from Qiagen using there suggested “Preparation of miRNA-enriched fractions separate from larger RNAs (>200)” protocol. The frozen tissue was placed in QIAzol lysis reagent and homogenized in a final volume of 18 μl. Two μl sample was used in a ligation reaction with 2 μl (1 μM) Universal1-CP1 capture probe oligonucleotide. Following ligation the ligation-mix were diluted in H₂O (Brain 50×, Liver 5000×) and 2 μl of this dilution was used as input in a EvaGreen ddPCR reaction (as described in the Materials and Methods section) with the primers O13-p1 and CP1-p1 at 56.6° C. FIG. 11 displays the result from this experiment. FIG. 11A displays the fluorescent signals in each droplet generated from the 8 samples (two control mice, two LNA oligonucleotide treated mice in both brain and liver tissue). As shown positive droplets in the PCR reaction originating from the LNA oligonucleotide treated mice was clearly visible. We saw only very few positive droplets in the control mice PCRs showing that only limited background noise PCR had occurred. We saw that the concentration of LNA oligonucleotide O13 in the liver (5000× dilution) was much higher than in the brain (50× dilutions) as expected. FIG. 11B displays a bar-chart with the number of events/positive droplets. The liver sample was initially also run in a 50× dilution, but all droplets came out positive (data not shown), and therefore the samples were diluted 5000×. Taken together we conclude that we have detected LNA oligonucleotide O13 in both mouse liver and brain, with much higher concentration observed in the liver (greater than 5000×). We saw an approximately 1% background noise in brain samples of the two control mice and no background in the control liver samples.

### Material and Methods:

### LNA Oligoes:

The used LNA oligo are displayed in table 1. A mix pool of LNAs (LNA-mix-pool1) was prepared by mixing the following LNA oligos. (O5 10 μM, O6 5 μM, O7 10 μM, O8 10 μM, O9 10 μM, O10 10 μM, O11 10 μM, O12 10 μM, O13 10 μM, O14 10 μM). Oligo 6(O6) is present in the mix in half the molar ratio of all the other LNAs. For ease of writing this mix pool will always be presented as an equimolar mix in the examples with the conc. being correct for all but O6 that always will be present in half the described concentration.

### LNA-DNA Ligation for PCR:

All Ligation reaction before PCR reaction was performed as follows: 2 μl Sample was added to 2 μl Capture probe mixed and incubated at 55° C. for 5 min. A mix containing 2 μl T4 DNA ligase (Thermo Scientific), 2 μl T4-DNA ligase buffer, 8 μl PEG and 4 μl H₂O was added to each tube and mixed. The following program was run on a thermal cycler. 2 min 37° C., 3 min 30° C., 5 min 22° C., 30 min 16° C. this cycle was repeated twice, then 10 min at 70° C. and stable at 4° C.

Sybr Green qPCR:

Sybr Green qPCR was performed using the SYBR® Green SuperMix low Rox kit from Quantabio. All reaction was performed 10 μL with the following setup: 5 μl SYBR® Green SuperMix, 100 nM forward primer, 100 nM reverse primer, 2 μl input template and H₂O up to 10 μL.

SYBR Green PCR Program:

| | |
|---|---|
| Hot start: | 95° C. 5 min |
| 40x cycles of: | |
| Denaturation: | 95° C. 10 sek |
| Anneling/extention: | (variable) ° C. 30 sek |
| Melt curve: | |
| Denaturation | 95° C. 15 sek |
| Anneling | 60° C. 1 min |
| Detection of duplex melting | 60° C.->95° C. 0.05° C./s |

ddPCR:

qLNA-PCR was performed with droplet digital PCR (emulsion PCR) using BioRad Automatic Droplet Generator (AutoDG) together with the OX200 droplet digital PCR system. The emulsion PCR was performed with QX200™ ddPCR™ EvaGreen Supermix and the Automated Droplet Generation Oil for EvaGreen. The PCR reaction that was used as input for the AutoDG was setup as follows: 11 μl ddPCR™ EvaGreen Supermix, forward primer (final conc. 100 nM), reverse primer (final conc. 100 nM), sample 2 μL and H₂O up to a total of 22 μL.

Following droplet generation the plate was sealed and run on the ddPCR program on a thermal cycler:

EvaGreen ddPCR Program:

| | |
|---|---|
| Hot start: | 95° C. 5 min |
| 40x cycles of: | |
| Denaturation: | 95° C. 30 sek |
| Anneling/extention: | (variable) ° C. 30 sek |
| Droplet stabilization: | 4° C. 5 min |
| | 90° C. 5 min |
| Hold: | 4° C. inf. |

Droplets were read on a QX200 droplet reader and the threshold was set manually.

In vivo qLNA-PCR study: LNA-mix-pool1 was injected IV in 2 adult mice with 950 nmol/kg in total (100 nmol/kg of each oligo and 50 nmol/kg of O6). 2 mice were injected with pure PBS as control. 7 days after injection the mice were sacrificed and various tissues were harvested and snap frozen with dry ice. Small RNA was purified from 50 mg tissue or 25 mg (liver tissue) using the miRNeasy kit from Qiagen using there suggested “Preparation of miRNA-enriched fractions separate from larger RNAs (>200)” protocol. The frozen tissue was placed in QIAzol lysis reagent and homogenized.

### Example 11: Determination of the Effect of Phosphorothioate Chirality of a Modified Oligonucleotide as a T4 DNA Ligase Substrate

### Materials and Methods

### Substrate Preparation:

One μl of 10 μM of the oligo O16, O17, O18, O19, O20, O21, O22, O8 or H₂O was mixed with 1 μl of 100 μM O8-CP1. Mixing of LNA oligonucleotide with the capture probe was followed by incubation at 50° C. for 5 min and placing on ice.

### Prepare Master Mix:

Master mix was prepared by combining 3 volumes of 50% PEG 4000, 3 volumes of H₂O, 1 volume of 10× T4 DNA Ligase Buffer (Thermo Fisher Scientific) and 1 volume of 30 U/μl T4 DNA Ligase HC (Thermo Fisher Scientific, catalog number EL0021).

### Perform Ligation Reaction:

Ligation reactions were initiated by transferring 8 μl of the appropriate master mix to the prepared substrate and incubating for (2 min at 37° C., 3 min at 30° C., 5 min at 22° C., 30 min at 16° C.)×3, kept at 4° C.

To each of the above mentioned reactions an equal volume of 2× Novex® TBE-Urea Sample Buffer (Thermo Fisher Scientific) has been added and samples were heat denatured for 2 min at 95° C. and placed on ice. Ten μl of thus prepared samples have been loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System(Bio Rad) on a Blue Tray.

### Results (FIG. 17):

This experiment is design to evaluate the importance of the chirality of the phosphotioate backbone with respect to T4 DNA Ligase ability to ligate the LNA oligo and the DNA capture probe together. We use fully stereo defined compounds of 08 as substrate. The chirallity of the last three phophotioate bindings of the 3′ end are indicated in the figure. The band of the ligated product appears just above the strong band of the capture probe (see FIG. 17). We saw that only LNA oligo nucleotides with an Rp conformation of the most 3′ phophotiate binding was able to efficiently ligate to the capture probe. Furthermore we see that it is only the chirality of last binding that is important for the effect of the ligation.

1. A capture probe oligonucleotide, for use in PCR or sequencing of a nucleoside modified oligonucleotide, comprising 5′-3′:
i) a first nucleotide segment comprising:
a. at least 3 5′ contiguous nucleotides of predetermined sequence (region 1A), wherein the 5′ most nucleotide is a DNA nucleotide with a terminal 5′ phosphate group, and
b. optionally a region of degenerate or predetermined nucleotides, positioned 3′ of region 1A (region 1B), and
c. a 3′ region which comprises a universal primer binding site (region 1C); and
ii) a second nucleotide segment, comprising:
a. a contiguous sequence of nucleotides which are complementary to the predetermined sequence 1A of the first nucleotide segment (region 2A), and
b. a region of at least 2 nucleotides, wherein the 3′ most nucleotide is a terminal nucleotide with a blocked 3′ terminal group (region 2B); wherein the first and second regions are covalently linked via a non-hybridizing linker moiety.

2. The capture probe oligonucleotide of claim 1, wherein the non-hybridizing linker moiety is selected from the group consisting of an alkyl linker, a polyethylene glycol linker, a non nucleosidic carbohydrate linker, a photocleavable linker (PC spacer), an alkyl disulfide linker, a region of 1,2-dideoxy ribose or abasic furan, or a region of nucleosides which comprise non-hybridising base groups.

3. The capture probe oligonucleotide of claim 1, wherein region 1A comprises at least 2 or at least 3 contiguous DNA nucleotides.

4. The capture probe oligonucleotide of claim 1, wherein the oligonucleotide comprises region 1B, wherein region 1B comprises a region of at least 3-30 degenerate nucleotides.

5. The capture probe oligonucleotide of any one of claim 1, wherein the oligonucleotide comprises region 1B, wherein region 1B comprises a region of at least 3-30 predetermined nucleotides.

6. The capture probe oligonucleotide of claim 1, wherein region 2A comprises DNA nucleotides which are complementary to region 1A.

7. The capture probe oligonucleotide of claim 1, wherein region 2B comprises a region of at least 2 nucleotides which are complementary to the 3′ nucleotides of the nucleoside modified oligonucleotide.

8. (canceled)

9. The capture probe oligonucleotide according to claim 1, wherein the 3′ terminal group on region 2B is either a nucleotidic modification which does not comprise a 3′-OH group, such as a modification selected from the group consisting of 3′deoxyribose, 2′, 3′-dideoxyribose, 1′, 3′-dideoxyribose, 1′, 2′, 3′-trideoxyribose, an inverted ribose, a 3′ phosphate, 3′ amino, 3′ labels such as 3′ biotin, and a 3′fluorophore; or a non-nucleosidic modification, such as a non-nucleosidic modification selected from the group consisting of a non-ribose sugar, an abasic furan, a linker group, an alkyl linker, a polyethylene glycol linker, a non nucleosidic carbohydrate linker, a photocleavable linker (PC spacer), an alkyl disulfide linker, a region of 1,2-dideoxy ribose or abasic furan, or a region of nucleosides which comprise non-hybridising base groups, a thiol modifier (eg. C6SH, C3SH), an amino modifier, glycerol, a conjugate, or a label.

10. Use of the capture probe oligonucleotide according to claim 1, for detecting, quantifying, sequencing, amplifying or cloning a nucleoside modified oligonucleotide.

11. A method for detecting, quantifying, sequencing, amplifying or cloning a nucleoside modified oligonucleotide in a sample, said method comprising the steps of:
a. Optionally performing an RNase treatment of the sample,
b. Admixing a capture probe oligonucleotide according to claim 1 with the sample under conditions which allow hybridization of the capture probe oligonucleotide to the nucleoside modified oligonucleotide,
c. Performing T4 DNA ligase mediated ligation of the 5′ terminus of the capture probe oligonucleotide and a 3′ terminus of the nucleoside modified oligonucleotide;
d. Adding a universal primer which is complementary to the capture probe oligonucleotide,
e. Performing 5′-3′ chain elongation of the universal primer, and
f. Detecting, quantifing, sequencing, amplifying or cloning a chain elongation product obtained in step e).

12. The method according to claim 11, wherein in step b, region 2B of the capture probe oligonucleotide hybridizes to the 3′ region of the nucleoside modified oligonucleotide, and wherein the universal primer of step d is complementary to region 1A of the capture probe oligonucleotide.

13. The method according to claim 11, wherein step f comprises a PCR amplification of the chain elongation product, and optionally cloning and/or sequencing the elongation product.

14. A method for identifying a nucleoside modified oligonucleotide which is enriched in a target cell or tissue in a mammal, said method comprising:
a. Administering a mixture of nucleoside modified oligonucleotides with different nucleobase sequences to a mammal,
b. Allowing the nucleoside modified oligonucleotides to be distributed within the mammal, for a period,
c. Isolating a population of nucleoside modified oligonucleotides from the target cell or tissue of the mammal, and
e. Identifying nucleoside modified oligonucleotides which are enriched in the target cell or tissue of the mammal.

15. The use of T4DNA ligase to ligate the 3′ terminus of a nucleoside modified oligonucleotide to the 5′ terminus of a DNA oligonucleotide, wherein the 3′ nucleoside of the nucleoside modified oligonucleotide is a LNA nucleoside.

16. The capture probe oligonucleotide of claim 4, wherein region 1B comprises DNA nucleotides.

17. The capture probe oligonucleotide of claim 5, wherein region 1B comprises DNA nucleotides.

18. The method according to claim 13, wherein step f comprises qPCR amplification.

19. The method according to claim 14, wherein the period of allowing the nucleoside modified oligonucleotides to be distributed within the mammal, is at least 24 hours.

20. The method according to claim 19, wherein the period of allowing the nucleoside modified oligonucleotides to be distributed within the mammal, is at least 48 hours.

21. The method according to claim 14, wherein the step of identifying nucleoside modified oligonucleotides which are enriched in the target cell or tissue of the mammal includes the steps of:
a. optionally performing an RNase treatment of the population of nucleoside modified oligonucleotides from the target cell or tissue of the mammal,
b. admixing a capture probe oligonucleotide with the sample under conditions which allow hybridization of the capture probe oligonucleotide to the nucleoside modified oligonucleotide,
c. performing T4 DNA ligase mediated ligation of a 5′ terminus of the capture probe oligonucleotide and a 3′ terminus of the nucleoside modified oligonucleotide;
d. adding a universal primer which is complementary to the capture probe oligonucleotide,
e. performing 5′-3′ chain elongation of the universal primer, and
f. detecting, quantifying, sequencing, amplifying or cloning a chain elongation product obtained in step e).


SEQUENCE LISTING (31 sequences)

SEQ ID NO: 1 - DNA, 22 bp, artificial
  Capture probe a1 sequence
tggaattctcgggtgccaagga

SEQ ID NO: 2 - DNA, 32 bp, artificial
  Capture probe A2 sequence
  misc_feature (1)..(1): n is a, c, g, or t
  misc_feature (7)..(10): n is a, c, g, or t
nwtrysnnnntggaattctcgggtgccaagga

SEQ ID NO: 3 - DNA, 22 bp, artificial
  Capture probe A3 sequence
tggaattctcgggtgccaagga

SEQ ID NO: 4 - DNA, 49 bp, artificial
  Capture probe A4 sequence - note iSP18 linker instertion between nts 34 and 35
  misc_feature (10)..(12): n is a, c, g, or t
  misc_feature (44)..(49): n is a, c, g, or t
ctatccagcnnntggaattctcgggtgccaaggagctggatagnnnnnn

SEQ ID NO: 5 - DNA, 49 bp, artificial; identical to SEQ ID NO: 4
  Capture probe A5 sequence - note first 4 5′ are RNA nucleotides, and a iSP18 linker positioned between nucleotides 34 and 35.
  misc_feature (10)..(12): n is a, c, g, or t
  misc_feature (44)..(49): n is a, c, g, or t

SEQ ID NO: 6 - DNA, 49 bp, artificial; identical to SEQ ID NO: 4
  Capture probe A6 sequence - note first 4 5′ are RNA nucleotides, last 15 nucleotides are RNA nucleotides, and a iSP18 linker positioned between nucleotides 34 and 35.
  misc_feature (10)..(12): n is a, c, g, or t
  misc_feature (44)..(49): n is a, c, g, or t

SEQ ID NO: 7 - DNA, 49 bp, artificial; identical to SEQ ID NO: 4
  Capture probe A7 sequence - note last 15 nucleotides are RNA nucleotides, and a iSP18 linker positioned between nucleotides 34 and 35.
  misc_feature (10)..(12): n is a, c, g, or t
  misc_feature (44)..(49): n is a, c, g, or t

SEQ ID NO: 8 - DNA, 47 bp, artificial
  Capture probe A8 sequence - note iSP18 linker positioned between nucleotides 32 and 33.
acgtccagcatctgttcaattggtacaaactggctggacgtaattca

SEQ ID NO: 9 - DNA, 47 bp, artificial
  Capture probe A9 sequence - note iSP18 linker positioned between nucleotides 32 and 33.
acgtccagcatctgttcaattggtacaaactggctggacgtgacagt

SEQ ID NO: 10 - DNA, 47 bp, artificial
  Capture probe A10 sequence - note iSP18 linker positioned between nucleotides 32 and 33.
  misc_feature (42)..(47): n is a, c, g, or t
acgtccagcatctgttcaattggtacaaactggctggacgtnnnnnn

SEQ ID NO: 11 - DNA, 66 bp, artificial
  Capture probe A11 sequence - note iSP18 linker positioned between nucleotides 57 and 58.
cggaccagcaagcttagagatcacggtatccagattcgctcatagtacacaactgcctccgacagg

SEQ ID NO: 12 - DNA, 20 bp, artificial
  primer sequence
tgtgctattctgtgaattac

SEQ ID NO: 13 - DNA, 18 bp, artificial
  primer sequence
tgtgctattctgtgaatt

SEQ ID NO: 14 - DNA, 15 bp, artificial
  primer sequence
gttgacactgtcacg

SEQ ID NO: 15 - DNA, 20 bp, artificial
  primer sequence
cagtttgtaccaattgaaca

SEQ ID NO: 16 - DNA, 23 bp, artificial
  primer sequence
aggaaagcaaacagtatatacgt

SEQ ID NO: 17 - DNA, 10 bp, artificial
  modified oligonucleotide sequence
ctgataagct

SEQ ID NO: 18 - DNA, 14 bp, artificial
  modified oligonucleotide sequence
agcattggtattca

SEQ ID NO: 19 - DNA, 18 bp, artificial
  modified oligonucleotide sequence
ctcactcggtcacgagtg

SEQ ID NO: 20 - DNA, 16 bp, artificial
  modified oligonucleotide sequence
  misc_feature (1)..(16): n is a, c, g, or t
nnnnnnnnnnnnnnnn

SEQ ID NO: 21 - DNA, 12 bp, artificial
  modified oligonucleotide sequence
gttgacactgtc

SEQ ID NO: 22 - DNA, 18 bp, artificial
  modified oligonucleotide sequence
tgtgctattctgtgaatt

SEQ ID NO: 23 - DNA, 16 bp, artificial
  modified oligonucleotide sequence
ttgaataagtggatgt

SEQ ID NO: 24 - DNA, 13 bp, artificial
  modified oligonucleotide sequence
gcaagcatcctgt

SEQ ID NO: 25 - DNA, 14 bp, artificial
  modified oligonucleotide sequence
actcgtacttccga

SEQ ID NO: 26 - DNA, 15 bp, artificial
  modified oligonucleotide sequence
agttataatccagct

SEQ ID NO: 27 - DNA, 16 bp, artificial
  modified oligonucleotide sequence
tgtatcgactgcatta

SEQ ID NO: 28 - DNA, 16 bp, artificial
  modified oligonucleotide sequence
cgtcagtatgcgaatc

SEQ ID NO: 29 - DNA, 19 bp, artificial
  modified oligonucleotide sequence
aggaaagcaaacagtatat

SEQ ID NO: 30 - DNA, 12 bp, artificial
  modified oligonucleotide sequence
gttgacactgtc

SEQ ID NO: 31 - DNA, 16 bp, artificial
  modified oligonucleotide sequence
  misc_feature (1)..(16): n is a, c, g, or t
nnnnnnnnnnnnnnnn